(19) United States
(12) Patent Application Publication
(10) Pub. No.: US 2009/0136506 A1
(43) Pub. Date: May 28, 2009

(54) Conserved Membrane Activator of Calcineurin (CMAC), a Novel Therapeutic Protein and Target

(76) Inventors: Mark Bittinger, West Roxbury, MA (US); Christine Chow, Boston, MA (US); Danilo Guerini, Reinach (CH); Mark Aron Labow, Lexington, MA (US); Brian Jude Latario, Groton, MA (US); Zhao-Hui Xiong, Winchester, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/089,005

(22) PCT Filed: Oct. 2, 2006
(86) PCT No.: PCT/US06/38482

§ 371 Date: Apr. 2, 2008

Related U.S. Application Data
(60) Provisional application No. 60/723,181, filed on Oct. 3, 2005.

(51) Int. Cl. A61K 39/395; C07K 14/00; C07K 16/00; C07H 21/00; A61K 31/7088; A61P 25/00; G01N 33/53; C12N 15/74; C12N 5/10; C12P 21/00

(52) U.S. Cl. 424/139.1; 530/324; 530/387.9; 536/23.53; 435/320.1; 435/325; 435/69.1; 514/44; 435/7.1

(57) ABSTRACT

The invention discloses the first known function and biological activity of the hypothetical protein MGC14327, now designated cMAC, which is herein identified as an important controller of T-cell activation. It is contemplated herein that cMAC is a suitable drug target for the development of new therapeutics to treat cMAC-associated disorders. The invention relates to methods to treat said pathological conditions and to pharmaceutical compositions therefore. The pharmaceutical compositions comprise modulators with inhibitory or agonist effect on cMAC protein activity and/or cMAC gene expression. The invention also relates to methods to identify compounds with therapeutic usefulness to treat said pathological conditions, comprising identifying compounds that can inhibit or agonize cMAC protein activity and/or cMAC gene expression.

### BACKGROUND OF THE INVENTION

There is a great deal of evidence to support the notion that T-lymphocytes are required for progression of systemic autoimmune disease. Simple reduction in T-cell numbers reduces the host immune response and improves survival in animal models of autoimmune disease. For instance, in a spontaneous murine model of lupus, the reduction in T cells with anti-T-cell antibodies reduced circulating T-cells, lowered autoantibody concentrations, reduced renal complications and improved animal survival (Wofsy D, Ledbetter J A, Hendler P L, Seaman W E. (1985) Treatment of murine lupus with monoclonal anti-T cell antibody. J. Immunol. February; 134(2):852-7).

Antibodies which block T-cell co-stimulatory receptors are also effective in prolonging animal survival (Finck B K, Linsley P S, Wofsy D. (1994) Treatment of murine lupus with CTLA4Ig Science. August 26; 265(5176):1225-7). Drugs which reduce T-cell proliferation or activation are effective in treating lupus in humans (cyclophosphamide, mycophenolate mofetil and cyclosporine A). Further supporting evidence that T-cells are involved in autoimmune disease in humans comes from the observation that lupus patients with HIV infections may experience remission due to the reduction of CD4+ T-cells (Byrd V M, Sergent J S. (1996) Suppression of systemic lupus erythematosus by the human immunodeficiency virus. J. Rheumatol. July; 23(7):1295-6.)

T lymphocytes also play an important role in acute graft rejection. Acute graft rejection is classically preceded by the migration of T cells into the graft, the clonal expansion of the activated T-cells, the proliferation of cytotoxic T-cells and subsequent tissue destruction and the loss of the graft. The ability of athymic mice to indefinitely accept grafts from other species demonstrates the particular importance T-cells play in graft rejection. Likewise, drugs which block T-cell responses or eliminate T-cells altogether are effective in preventing graft rejection in humans (Sykes M, Auchincloss H, Sachs D H (2003) “Transplantation immunology”, in Fundamental Immunology, ed W E Paul, Lippincott Williams & Wilkins, Philadelphia, p. 1499.)

The activation of naïve T-cells requires stimulation of the T-cell receptor (TCR) and a co-stimulatory receptor (CD28). TCR/CD28 engagement activates a complex signaling cascade which causes an increase in intracellular calcium through the release of intracellular calcium stores and subsequent influx of calcium through the CRAC (calcium release activated calcium current) channel. The increase in intracellular calcium results in the activation of calcineurin which dephosphorylates NFAT. NFAT proteins are a family of transcription factors that once dephosphorylated are translocated into the nucleus where they drive transcription of one of the most important lymphokines in cellular rejection, IL-2 (Hutchinson I, (2001) “Transplantation and rejection” in Immunology, I Roitt, J Brostoff and D Male, Mosby New York p. 389.). IL-2 stimulates the proliferation of cytotoxic T-cells which release cytolytic molecules, performin and granzyme, that mediate the destruction of the graft. The immunosuppressive drug cyclosporine A inhibits the phosphatase enzyme calcineurin which prevents the translocation of NFAT into the nucleus and thus prevents the transcription of IL-2.

The TORC proteins are CREB co-activators which, like NFAT, are translocated into the nucleus in response to the mobilization of intracellular calcium stores. TORC is believed to facilitate CRE mediated gene expression. Proteins which regulate NFAT and/or TORC, may be suitable targets for therapeutic intervention. Applicants report herein that cMAC is a potent regulator of T-cell activation and regulates NFAT and TORC nuclear translocation.

### SUMMARY OF THE INVENTION

The instant disclosure relates to the discovery that a protein of previously unknown function, referred to herein as cMAC (“conserved Membrane Activator of Calcineurin”), is a potent regulator of T-cell activation, and is involved in calcium-mediated nuclear translocation of NFAT and TORC1. As such, cMAC is an important therapeutic protein and therapeutic target for the treatment of cMAC-associated diseases (defined herein), using small molecules, antibodies, nucleic acids and other therapeutic agents which modulate cMAC activity or expression.

In one aspect the invention relates to mature or native cMAC polypeptide. Accordingly, the invention relates to the isolated polypeptide of SEQ ID NO: 2, or a fragment thereof, or a substantially similar protein sequence having sequence identity of at least 50% with SEQ ID NO: 2, or a functional equivalent thereof, and exhibiting a biological activity selected from ion transport, ion diffusion, calcineurin pathway activation, calcium dependent activation of a T-cell, nuclear translocation of TORC, nuclear translocation of NFAT or cAMP Response Element (CRE)-driven gene expression activity of native SEQ ID NO: 2.

In other aspects, the invention comprises an isolated nucleic acid molecule encoding cMAC, a vector comprising the nucleic acid molecule, preferably an expression vector comprising the nucleic acid molecule operably linked to a promoter, a host cell comprising the vector molecule, including mammalian and bacterial host cells, and a method of using a nucleic acid molecule encoding cMAC to effect the production of cMAC, comprising culturing a host cell comprising the vector molecule.

Another aspect of the invention provides an antibody or antibody fragment that is capable of binding the a cMAC polypeptide of the invention. A further aspect of the invention relates to an RNAi agent capable of downregulating expression of cMAC, preferably such RNAi agent comprises at least one nucleic acid selected from Table 5 or Table 6.

Other aspects of the invention relates to the use of an antibody or RNAi agent according to the invention for the manufacture of a medicament for the treatment of a cMAC-associated disorder (as defined herein) and to a method of treating a disorder in a subject comprising administering to the subject an effective amount of an agent that modulates the amount or activity of cMAC. Accordingly, the invention comprises the use of an antibody, an antibody fragment or a polypeptide comprising a cMAC-specific binding region in the treatment of a disorder in a subject, especially wherein the disorder is a cMAC-associated disorder. Alternatively, the invention comprises use of an RNAi agent or siRNA specific for cMAC in the treatment of a disorder in a subject, especially wherein the disorder is a cMAC-associated disorder.

In various aspects, this method comprises administering an agent which inhibits cMAC, wherein the disorder is a cMAC-associated disorder (as defined herein), or wherein the agent is an antibody, an antibody fragment or a polypeptide containing a cMAC-specific binding region. Optionally said agent is administered as a pharmaceutical composition.

In another aspect, this method comprises administering to the subject an effective amount of an agent that inhibits the expression of cMAC. Such agent includes an inhibitory nucleic acid capable of specifically inhibiting expression of cMAC. Various embodiments include that said inhibitory nucleic acid is selected from among the group consisting of an antisense oligonucleotide, an RNAi agent, and a ribozyme, dsRNA, siRNA, and shRNA. Optionally said agent is administered as a pharmaceutical composition.

In another aspect, the invention comprises a method of treating a disorder in a subject comprising administering to the subject an effective amount of an agent that enhances the activity of cMAC. Such method includes a method comprising administering to the subject an effective amount of an agent that increases the expression of cMAC, for example where the agent is a gene therapy vector comprising a nucleic acid encoding cMAC or a fragment thereof, or where the agent is an enhancer of cMAC gene transcription.

In terms of compositions, the invention comprises an antibody or antibody fragment that binds specifically to cMAC (SEQ ID NO.2), and any polypeptide comprising a cMAC-specific binding region. Such antibody includes an antibody fragment which is an Fab or F(ab′)2 fragment, or wherein the antibody is a monoclonal antibody. The invention includes a pharmaceutical composition comprising an effective amount of an agent which inhibits the expression of cMAC or inhibits an activity of cMAC, and a pharmaceutically acceptable carrier. Such agent may be an antisense oligonucleotide, an RNAi agent, an antibody fragment which binds specifically to cMAC, or a polypeptide comprising a cMAC-specific binding region. In a preferred embodiment, the pharmaceutical composition comprises an antibody or antibody fragment that binds specifically to cMAC (SEQ ID NO.2), or any polypeptide comprising a cMAC-specific binding region that binds to an epitope of cMAC selected from among SEQ ID NOs. 6, 7, 8, 9, 10.

The invention also comprises a method of treating a disorder in a subject comprising administering to the subject an effective amount of a pharmaceutical composition of an agent that inhibits the activity of cMAC, especially wherein the disorder is a cMAC-associated disorder, and wherein said agent is an antibody or fragment thereof which binds specifically to cMAC (SEQ ID NO:2) or a polypeptide comprising a cMAC-specific binding region. Such agent optionally binds to an epitope of cMAC selected from among SEQ ID NO. 6, 7, 8, 9, 10.

The invention further comprises screening assay methods for identifying a compound useful for the treatment of a cMAC-associated disorder comprising (a) contacting a test compound with cMAC; and (b) detecting a change of a biological activity of cMAC compared to cMAC not contacted with the test compound, wherein detecting a change identifies said test compound as useful for the treatment of said disorder. Similarly the invention comprises screening assay methods for identifying a compound useful for treatment of a cMAC-associated disorder comprising: (a) contacting a test compound with cMAC under sample conditions permissive for cMAC biological activity; (b) determining the level of a cMAC biological activity; (c) comparing said level to that of a control sample lacking said test compound; and, (d) selecting a test compound which causes said level to change for further testing as a potential agent for treatment of said disorder. The invention comprises a method for testing if a compound modulates a cMAC biological activity comprising: (a) contacting a test compound with cMAC; and (b) detecting a change of a biological activity of cMAC compared to cMAC not contacted with the test compound, wherein detecting a change identifies said test compound as a modulator of cMAC biological activity. Likewise, the invention comprises a method to identify modulators useful to treat a disorder comprising assaying for the ability of a candidate modulator to inhibit the activity of a cMAC protein; and a method to identify modulators useful to treat a disorder comprising assaying for the ability of a candidate modulator to inhibit the expression of a cMAC protein.

In these methods said change or said modulation may be a reduction or an increase of such biological activity. Further, said biological activity may be selected from among ion transport, ion diffusion, protein-cMAC interaction or cMAC modification, calcium dependent activation of a T-cell, nuclear translocation of TORC, and cAMP Response Element (CRE)-driven gene expression.

According to the invention, cMAC-related or cMAC associated disorders include, but are not limited to, autoimmune disease, immunosuppression, inflammatory disease, cancer, cardiovascular disease and neurological disease.

### DESCRIPTION OF THE FIGURES

FIG. 1 cMAC is a predicted integral membrane protein. Transmembrane domain prediction of the cMAC sequence of using the TMHMM algorithm. Two small predicted extracellular domains are located at amino acids 36-49 and 101-110.

FIG. 2 cMAC is a highly conserved protein. ClustalW alignment of vertebrate proteins with similarity to cMAC. Potential transmembrane helices predicted by the TMHMM algorithm are indicated by lines.

FIG. 3 cMAC mRNA levels as measured by Affymetrix expression profiling.

FIG. 4 cMAC over expression induces TORC translocation in HEK293 cells. Bittenger et. al. identified TRPV6 and PKA as hits in the TORC-eGFP translocation screen (Bittenger et. al. Curr Biol. 2004 Dec. 14; 14(23):2156-61). cMAC was also identified though not previously disclosed.

FIG. 5 cMAC mediated translocation of TORC1-eGFP is blocked by calcineurin inhibitor CsA. In panel A, HeLa:TORC1-eGFP cells were transduced with stop-codon virus (vector), or humanTRPV6, or human CMAC virus (50 uL). Panel B cells were treated as in A except cells were treated with 5 uM cyclosporin A (CsA) for 1 hour prior to fixing.

FIG. 6 cMAC induces NFAT-dependent transcription. HEK293 cells were co-transfected with an NFAT-luciferase reporter plasmid, transfection control and the following constructs: Empty vector (CMV), TRPV6 and cMAC and treated with either DMSO, 5 μM CsA, 10 μM PMA, or 10 μM PMA and 5 μM CsA.

FIG. 7 cMAC induces NFAT1 translocation in Jurkat cells. Panel A. Lentiviral mediated overexpression cMAC, control vector (translation stop sequence), and TRPV6 with and without cyclosporin A; B. Same treatment as in A except cells were PMA sensitized with PMA 6 hours prior to fixing.

FIG. 8 cMAC induces NFAT2 translocation in Jurkat cells. A. Viral (pLLB1-GW-Kan) mediated overexpression cMAC, control vector (translation stop sequence), TRPV6 with and without cyclosporin; B. Same treatment as in A except cells were PMA sensitized 6 hours prior to fixing.

FIG. 9 Murine cMAC and human homologue overexpression activates Jurkat T-cells. Jurkat cells were transduced with viral expression vector (QL-GW-final-Kan) containing negative control empty vector (translation stop sequence), TRPV6 calcium channel, and NM_{—}177244 (murine cMAC) and NM_{—}053045 (human cMAC). IL-2 protein (ELISA) and ICOS surface marker expression were measured 72 hours post transduction (48 hours post transduction cells were sensitized with PMA and anti TCR antibody).

FIG. 10 Multiple viral shDNA sequences targeting cMAC block TCR/CD28 activation of Jurkat T-cells. Cells were transduced with viral constructs (pLKO.1), selected with puromycin and activated with TCR/CD28 6 days post transduction. IL-2 protein levels were measured and normalized by the number of viable cells present in each well. The shDNA construct pGL3-Luc served as the negative control shDNA for viral transductions.

FIG. 11 Human CMAC: NM_{—}053045: Homo sapiens hypothetical protein MGC14327 (MGC14327), mRNA (gi|16596685|ref|NM_{—}053045.1|[16596685]) (SEQ ID NO: 1) and human hypothetical protein LOC94107 [Homo sapiens; >gi|16596686|ref|NP_{—}444273.1|.] (SEQ ID NO: 2).

FIG. 12 Human CMAC genomic promoter sequence (NM_{—}053045.1_{—}5′_-3000+100 NT_{—}024000.16 886093 882993) (SEQ ID NO: 3).

FIG. 13 Isolated nucleic acid sequence for the cMAC 5′UTR (SEQ ID NO: 4) and isolated nucleic acid sequence for the cMAC 3′UTR (SEQ ID NO: 5).

FIG. 14 Murine cMAC NM_{—}177344: Mus musculus RIKEN cDNA C730025P13 gene (C730025P13Rik), mRNA (gi|31340922|ref|NM_{—}177344.2|) (SEQ ID NO: 11) and >gi|18490941|gb|BC022606.1| Mus musculus RIKEN cDNA C730025P13 gene, mRNA (cDNA clone MGC:31129 IMAGE:4165766), complete cds (SEQ ID NO 12) and mouse cMAC amino acid sequence translated from NM_{—}177344.2 (SEQ ID NO: 13)

FIG. 15 Other orthologs of human cMAC from other species Mus musculus (SEQ ID NO: 14); Rattus norvegicus (SEQ ID NO: 15); Canis familiaris (SEQ ID NO: 16); Pan troglodytes (SEQ ID NO: 17); Xenopus tropicalis (SEQ ID NO: 18); Danio rerio (SEQ ID NO: 19); Gallus gallus (SEQ ID NO: 20); Branchiostoma floridae (SEQ ID NO: 21)

### DETAILED DESCRIPTION OF THE INVENTION

### Definitions

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and, M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art.

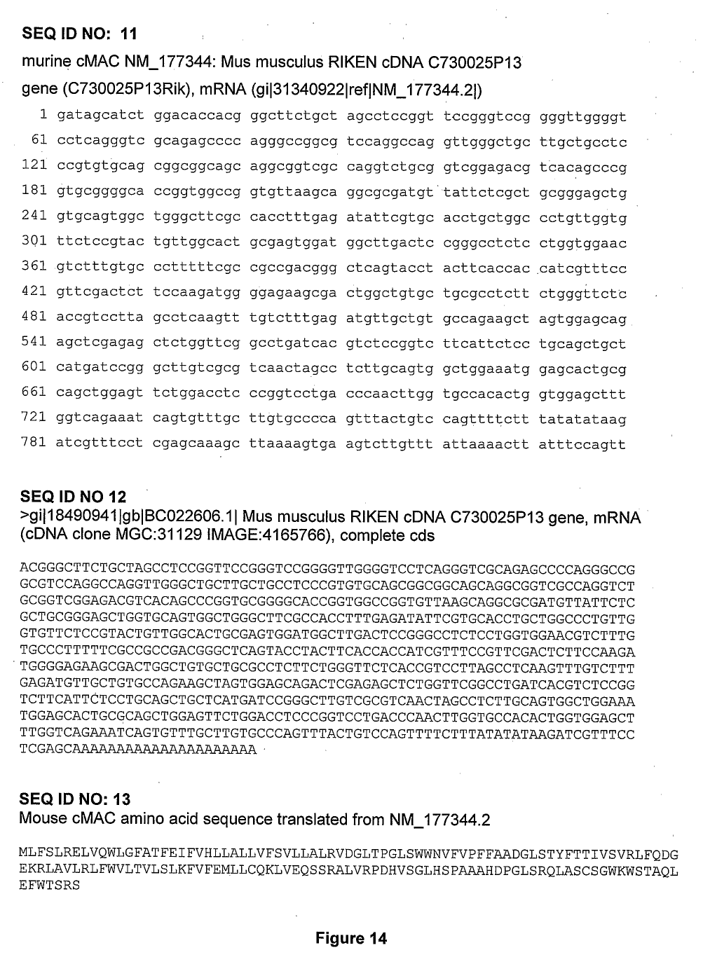

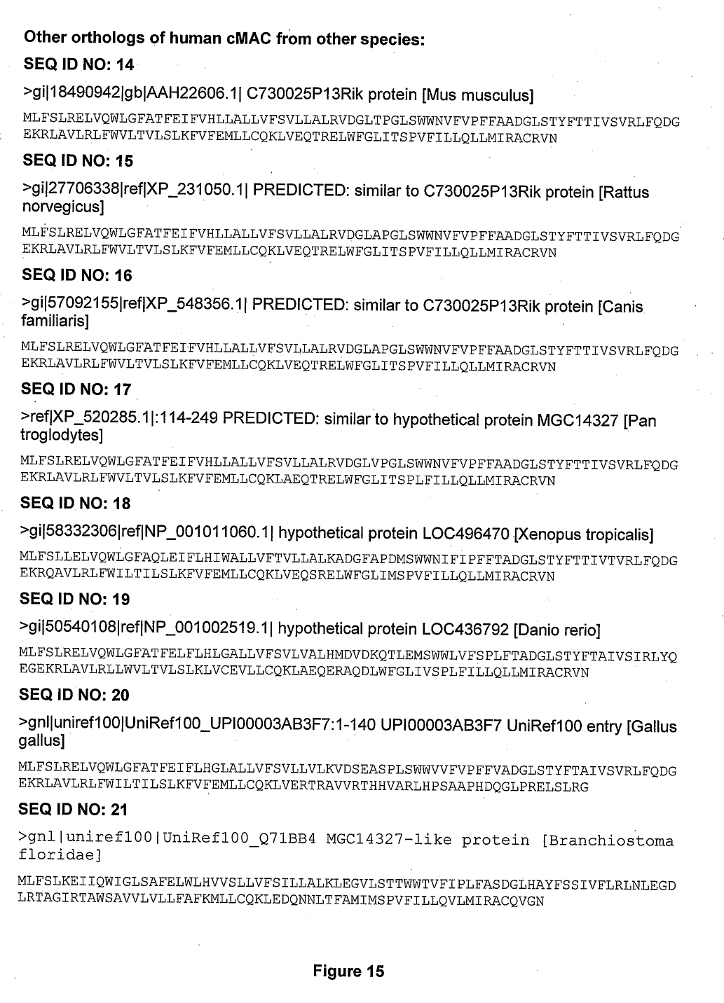

“cMAC” or “conserved Membrane Activator of Calcineurin” is the subject protein of the invention and described in detail below. The various names assigned to this protein and gene may be changed according to scientific usage. Consequently, the claims of this patent and content of this specification are intended to refer to the subject gene and protein of the invention, and its various fragments and forms, without regard to the specific name assigned. Thus, “cMAC” is defined herein to be any polypeptide sequence that possesses at least one biological property (as defined below) of a naturally occurring polypeptide comprising the polypeptide sequence of SEQ ID NO:2 or any one of the orthologs thereof shown in FIGS. 14 and 15.

A “cMAC-associated disorder” or “cMAC-related disorder” means a disorder that may be treated by modulating the activity of cMAC. These disorders include, but are not limited to, autoimmune disease, immunosuppression, inflammatory disease, cancer, cardiovascular disease and neurological disease.

Examples of autoimmune diseases include disorders and/or conditions including sarcoidosis, fibroid lung, idiopathic interstitial pneumonia, obstructive airways disease, including conditions such as asthma, intrinsic asthma, extrinsic asthma, dust asthma, particularly chronic or inveterate asthma (for example late asthma and airway hyperreponsiveness), bronchitis, including bronchial asthma, infantile asthma, allergic rheumatoid arthritis, systemic lupus erythematosus, nephrotic syndrome lupus, Hashimoto's thyroiditis, multiple sclerosis, myasthenia gravis, type I diabetes mellitus and complications associated therewith, type II adult onset diabetes mellitus, uveitis, nephrotic syndrome, steroid-dependent and steroid-resistant nephrosis, palmo-plantar pustulosis, allergic encephalomyelitis, glomerulonephritis, psoriasis, psoriatic arthritis, atopic eczema (atopic dermatitis), contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia areata, eosinophilic fasciitis, atherosclerosis, conjunctivitis, keratoconjunctivitis, keratitis, vernal conjunctivitis, uveitis associated with Behcet's disease, herpetic keratitis, conical cornea, dystorphia epithelialis corneae, keratoleukoma, ocular pemphigus, Mooren's ulcer, scleritis, Graves' opthalmopathy, severe intraocular inflammation, inflammation of mucosa or blood vessels such as leukotriene B4-mediated diseases, gastric ulcers, vascular damage caused by ischemic diseases and thrombosis, ischemic bowel disease, inflammatory bowel disease (e.g. Crohn's disease and ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sjogren' syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), B-virus hepatitis, non-A/non-B hepatitis and cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, amyotrophic lateral sclerosis or rheumatic fever.

Immunosuppression is desirable for treatment of acute or chronic graft rejection such as acute or chronic rejection of cells, tissue or solid organ allo- or xeno-grafts of e.g. pancreatic islets, stem cells, bone marrow, skin, muscle, corneal tissue, neuronal tissue, heart, lung, combined heart-lung, kidney, liver, bowel, pancreas, trachea or oesophagus. Treatment of graft-versus-host disease is also included. Chronic rejection is also named graft vessel diseases or graft vasculopathies.

Treatment of immunosuppression, in the sense of treating immune compromised subjects, may also be achieved by modulation of cMAC, such as may result from the activation of T-cells which are not sufficiently active in a subject to resolve the disease or condition. Diseases caused by deficient immune response include but are not limited to AIDS, SLE, and the like.

Inflammatory diseases which may be treated by modulation of cMAC include those inflammatory diseases, disorders and/or conditions thought to respond to CsA treatment.

Cancer includes but is not limited to neoplasia and abnormal cell growth associated with pre-cancerous or cancerous conditions. Those skilled in the art are familiar with the numerous forms of cancer, neoplasia and abnormal cell growth, in particular lymphoma, leukemia, and other hematological cancers.

Cardiovascular disease includes but is not limited to cardiovascular diseases, disorders and conditions such as cardiac hypertrophy and heart failure.

Neurological diseases and/or conditions include diseases, disorders and/or conditions including but are not limited to Alzheimer's Disease, Parkinsons's Disease and Huntington Disease, and include neuroprotection that may be achieved by methods and compositions for the modulation of cMAC.

While antagonists or inhibitors of cMAC are suggested for use in any or all of the above noted diseases, disorders and/or conditions, agonists of cMAC are particularly implicated and desirable for the treatment of cancer, diseases caused by deficient immune response and for neuroprotection.

“cMAC-associated disorder” is sometimes referred to herein as a “pathological condition” which is associated with abnormal cMAC expression, abnormal cMAC activity, or abnormal activation of T-cells.

As used herein, “disorder” includes a disease, disorder, or condition, whether existing or prognostically identified, and includes symptoms or side-effects of diseases, disorders or conditions and the pharmaceuticals used to treat them.

The ability of a substance to “modulate” a cMAC protein (e.g. a “cMAC modulator”) includes, but is not limited to, the ability of a substance to inhibit or enhance one or more biological activities of a cMAC protein and/or inhibit or enhance its expression. Such modulators include both agonists and antagonists of cMAC activity. Such modulation could also involve effecting the ability of other proteins to interact with CMAC, for example related regulatory proteins or proteins that bind to cMAC.

“Biological activity” when used in conjunction with either “isolated cMAC” or cMAC” means having an activity selected from ion transport activity, ion diffusion activity, a calcium dependent activation of a T-cell activity, nuclear translocation of TORC, nuclear translocation of NFAT or cAMP Response Element (CRE)-driven gene expression activity when compared to the activity or mature or native or endogenous cMAC of e.g. SEQ ID NO: 2.

The term “agonist”, as used herein, refers to a molecule (i.e. modulator) which, directly or indirectly, may modulate a polypeptide (e.g. a cMAC polypeptide) and which increases the biological activity of said polypeptide. Agonists may include proteins, nucleic acids, carbohydrates, organic molecules, small organic molecules (with or without inorganic moieties) or other molecules. A modulator that enhances gene transcription, biological activity or the biochemical function of a protein is something that increases transcription or stimulates the biochemical properties or activity of said protein, as the case may be.

The terms “antagonist” or “inhibitor” as used herein, refer to a molecule (i.e. modulator) which directly or indirectly modulates a polypeptide (e.g. the cMAC polypeptide) which blocks or inhibits the expression and/or the biological activity of said polypeptide. Antagonists and inhibitors may include proteins, nucleic acids, carbohydrates, or other molecules. A modulator that inhibits expression or the biochemical function of a protein is something that reduces gene expression or biological activity of said protein, respectively.

“Nucleic acid sequence”, as used herein, refers to an oligonucleotide, nucleotide or polynucleotide, and fragments or portions thereof, which polymeric components may be DNA, RNA, modified nucleotides, nucleotide mimetics or combinations thereof; and may be of genomic or synthetic origin, and may be single or double stranded, and represent the sense or anti-sense strand.

The term “antisense” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. The designation “negative” is sometimes used in reference to the antisense strand, and “positive” is sometimes used in reference to the sense strand.

The term “RNAi agent” as used herein, refers to compounds and compositions which can act through an RNA interference (or “RNAi”) mechanism (see, for general reference, He and Hannon, (2004) Nat. Genet. 5:522-532). RNAi agents such as short interfering RNA (“siRNA”), double stranded RNA (“dsRNA”), short hairpin RNA (“shRNA”, also sometimes called ‘synthetic RNA’) are commonly used, others are in development. When introduced into a cell or synthesized within a cell RNAi agents are incorporated into a macromolecular complex which uses strands of the RNAi agent to target and cleave RNA strands containing the complementary (or substantially complementary) sequence.

As contemplated herein, antisense oligonucleotides, triple helix DNA, RNA aptamers, RNAi agents such as siRNA, dsRNA, and shRNA, ribozymes and single stranded RNA are designed to inhibit cMAC expression such that the chosen nucleotide sequence of the inhibitory molecule is designed to cause inhibition of endogenous cMAC protein synthesis. For example, based on the disclosure herein, knowledge of the cMAC nucleotide sequence may be used to design an siRNA molecule which inhibits cMAC expression without undue experimentation. Similarly, ribozymes can be synthesized to recognize specific nucleotide sequences of a protein of interest and cleave it (Cech. J. Amer. Med. Assn. 260:3030 (1988)). Techniques for the design of such molecules for use in targeted inhibition of gene expression are well known to one of skill in the art.

The term “sample” or “biological sample” as used herein, is used in its broadest sense. A biological sample from a subject may comprise blood, urine or other biological material with which activity or gene expression of cMAC proteins may be assayed.

As used herein, the term “antibody” is interchangeable with “immunoglobulin” and refers to immunoglobulins of the general form found in vertebrate species including mammals such as humans, primates, rodents, rabbits, and many other species in which such immunoglobulins have been identified. In particular such immunoglobulins include the heavy chain antibodies, found in camelids, which lack light chains and as a result have variable domains that reflect the absence of a V_{L} partner. “Antibody” means molecules corresponding to complete immunoglobulins, as well as fragments thereof, such as Fa, F(ab′)₂, and Fv, which are capable of binding the epitopic determinant. The term “antibody fragment” refers more specifically to these fragments and or immunoglobulin-like polypeptides that do not comprise a complete immunoglobulin.

The term “humanized antibody” as used herein, refers to antibody molecules in which amino acids have been replaced in the non-antigen binding regions in order to more closely resemble a human antibody, while still retaining the original binding ability. Depending on context, this phrase may also include ‘primatized’ antibodies, wherein an antibody first obtained from a non-primate organism has been modified to more closely resemble a primate immunoglobulin.

A “polypeptide comprising a cMAC-specific binding region” means a polypeptide that incorporates one or more binding regions which bind specifically to the cMAC protein of SEQ ID NO: 2. A classic kind of antigen specific binding region is a complementarity determining region (“CDR”) found in an immunoglobulin. CDRs are short amino acid sequences which bind specifically to the antigen in question and provide the basis for selectivity of binding of the polypeptide in which it resides. CDRs are typically identified from immunoglobulins but can be generated by other means. CDRs were originally defined on immunoglobulins using common definitions such as the Kabat definition, the Chothia definition (based on the location of the structural loop regions); the AbM definition (a compromise between the two used by Oxford Molecular's AbM antibody modelling software); and the contact definition which is possibly the most useful for people wishing to perform mutagenesis to modify the affinity of an antibody since these are residues which take part in interactions with antigen. Antigen specific binding regions also include relatively short amino acid sequences that bind to an antigen, even if those sequences are not derived from CDRs. PCT publication WO 2004/044011, incorporated herein by reference, provides an example of how such antigen specific binding regions (which are not CDRs) can be identified and developed. Other methods are known to those skilled in the art. Once developed a cMAC-specific binding region may be employed in a wide variety of known and future frameworks or scaffolds, including any immunoglobulin isotype or fragment thereof, and other non-immunoglobulin framework or scaffold polypeptides (discussed elsewhere herein)) to provide antigen binding specificity. All such polypeptides are considered herein as “polypeptides comprising a cMAC-specific binding region”.

A peptide mimetic is a synthetically derived peptide or non-peptide agent created based on a knowledge of the critical residues of a subject polypeptide which can mimic normal polypeptide function. Peptide mimetics can disrupt binding of a polypeptide to its receptor or to other proteins and thus interfere with the normal function of a polypeptide. For example, a cMAC mimetic would interfere with normal cMAC function.

A “therapeutically effective amount” is the amount of drug sufficient to treat, prevent or ameliorate pathological conditions related to the function, activity or expression of cMAC.

To “treat” includes to prevent or ameliorate, as the context may imply, and includes such treatment whether intent is therapeutic, prophylactic, or directed to relief of symptoms only.

“Related regulatory proteins” and “related regulatory polypeptides” as used herein refer to polypeptides involved in the regulation of cMAC proteins which may be identified by one of skill in the art using conventional methods such as described herein.

Abnormal activation of T-cells can include excessive activation, e.g., states where the mRNA encoding cMAC protein is up-regulated or the cMAC protein has enhanced activity or amounts in a cell through either increases in absolute quantity or specific activity; abnormal activation may also include as well as states in which there is a down-regulation of cMAC gene expression, protein level or protein activity or there is abnormally low T-Cell activation.

“Subject”, when used in relation to receiving treatment, refers to any human or nonhuman organism.

In its broadest sense, the term “substantially similar” or “equivalent”, when used herein with respect to a nucleotide sequence, means a nucleotide sequence corresponding to a reference cMAC nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The percentage of identity between the substantially similar nucleotide sequence and the reference nucleotide sequence desirably is at least 80%, more desirably at least 85%, preferably at least 90%, more preferably at least 95%, 96%, 97%, or 98%, still more preferably at least 99%.

A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C., yet still encodes a functionally equivalent gene product. Generally, hybridization conditions may be highly stringent or less highly stringent. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). Suitable ranges of such stringency conditions for nucleic acids of varying compositions are described in Krause and Aaronson (1991), Methods in Enzymology, 200:546-556 in addition to Maniatis et al., cited above.

When used with respect to a polypeptide sequence, “substantially similar” means a protein sequence corresponding to a cMAC polypeptide disclosed herein, such protein sequence having substantially the same structure and function as the cMAC polypeptide, including isoforms, homologs, orthologs and modified sequences containing amino acid sequence identity across the length of the protein of at least 50%, 55%, 60%, 65%, 70%, 75%, 80%, 85%, 90%, 95% or 98%.

“Functionally equivalent,” as utilized herein, may refer to a protein or polypeptide capable of exhibiting a substantially similar in vivo or in vitro activity as the endogenous differentially expressed gene products encoded by the differentially expressed gene sequences described above. “Functionally equivalent” may also refer to proteins or polypeptides capable of interacting with other cellular or extracellular molecules in a manner substantially similar to the way in which the corresponding portion of the endogenous differentially expressed gene product would. For example, a “functionally equivalent” peptide would be able, in an immunoassay, to diminish the binding of an antibody to the corresponding peptide (i.e., the peptide the amino acid sequence of which was modified to achieve the “functionally equivalent” peptide) of the endogenous protein, or to the endogenous protein itself, where the antibody was raised against the corresponding peptide of the endogenous protein. An equimolar concentration of the functionally equivalent peptide will diminish the aforesaid binding of the corresponding peptide by at least about 5%, preferably between about 5% and 10%, more preferably between about 10% and 25%, even more preferably between about 25% and 50%, and most preferably between about 40% and 50%.

A “fragment” is a portion of a naturally occurring mature, native or endogenous full-length cMAC sequence having one or more amino acid residues deleted. The deleted amino acid residue(s) may occur anywhere in the polypeptide, including at either the N-terminal or C-terminal end or internally. Such fragment will have at least one biological property in common with cMAC. cMAC fragments typically will have a consecutive sequence of at least 10, 15, 20, 25, 30, 40, 50 or 60 amino acid residues that are identical to the sequences of the cMAC isolated from a mammal including cMAC of SEQ ID NO:2.

“Elevated transcription of mRNA” refers to a greater amount of messenger RNA transcribed from the natural endogenous human gene encoding a cMAC polypeptide of the present invention in an appropriate tissue or cell of an individual suffering from a pathological condition related to abnormal activation of cMAC gene expression or abnormal activation of T-cells compared to control levels, in particular at least about twice, preferably at least about five times, more preferably at least about ten times, most preferably at least about 100 times the amount of mRNA found in corresponding tissues in subjects who do not suffer from such a condition. Such elevated level of mRNA may eventually lead to increased levels of protein translated from such mRNA in an individual suffering from said condition as compared with a healthy individual.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and the like.

“Heterologous” as used herein means “of different natural origin” or represents a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g. a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.”

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” refers to DNA sequences, such as initiator sequences, enhancer sequences, and promoter sequences, which induce, repress, or otherwise control the transcription of protein encoding nucleic acid sequences to which they are operably linked.

As used herein, “human transcriptional control sequences” are any of those transcriptional control sequences normally found associated with a human gene encoding the cMAC protein of the present invention as it is found in the respective human chromosome.

As used herein, “non-human transcriptional control sequence” is any transcriptional control sequence not found in the human genome.

As used herein, a “chemical derivative” of a polypeptide of the invention is a polypeptide of the invention that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16th ed., Mack Publishing Co., Easton, Pa. (1980).

### Introduction

The instant invention is based on the surprising discovery that the cMAC (“conserved Membrane Activator of Calcineurin”) protein previously referred to in public sequence databases as “Homo sapiens hypothetical protein MGC14327 (MGC14327), mRNA. (GenBank Accession No. NM_{—}053045)” and heretofore of unknown function, is a potent regulator of NFAT and TORC1 nuclear translocation, functions via the Calcineurin pathway, and has an important role in T-cell activation (Table 1, FIG. 11).

TABLE 1

| Description | SEQ ID NO. |
|---|---|
| cDNA sequence of the gene encoding human cMAC | SEQ ID No. 1 |
| (GenBank Accession NM_053045). | |
| Full length amino acid sequence of the human | SEQ ID No. 2 |
| cMAC protein. | |

As used herein “cMAC” means, depending on context, a cMAC protein, or a nucleic acid comprising a sequence encoding the cMAC protein (e.g. the cMAC gene), or fragments or fusions thereof.

The cMAC protein disclosed herein includes the cMAC polypeptide identified herein, any and all forms of these polypeptides including, but not limited to, partial forms, homologs, isoforms, precursor forms, the full length polypeptides, fusion proteins containing the protein sequence, proteins which are substantially similar or equivalent to cMAC, or fragments of any of the above, from humans, primates, mammals, vertebrates, invertebrates or any other species.

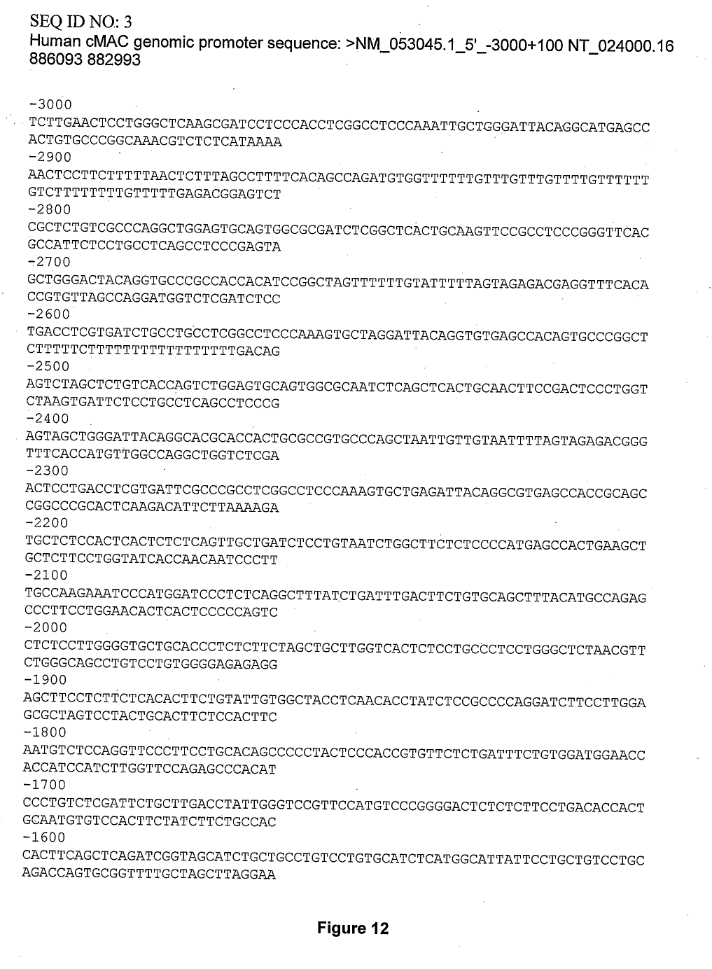

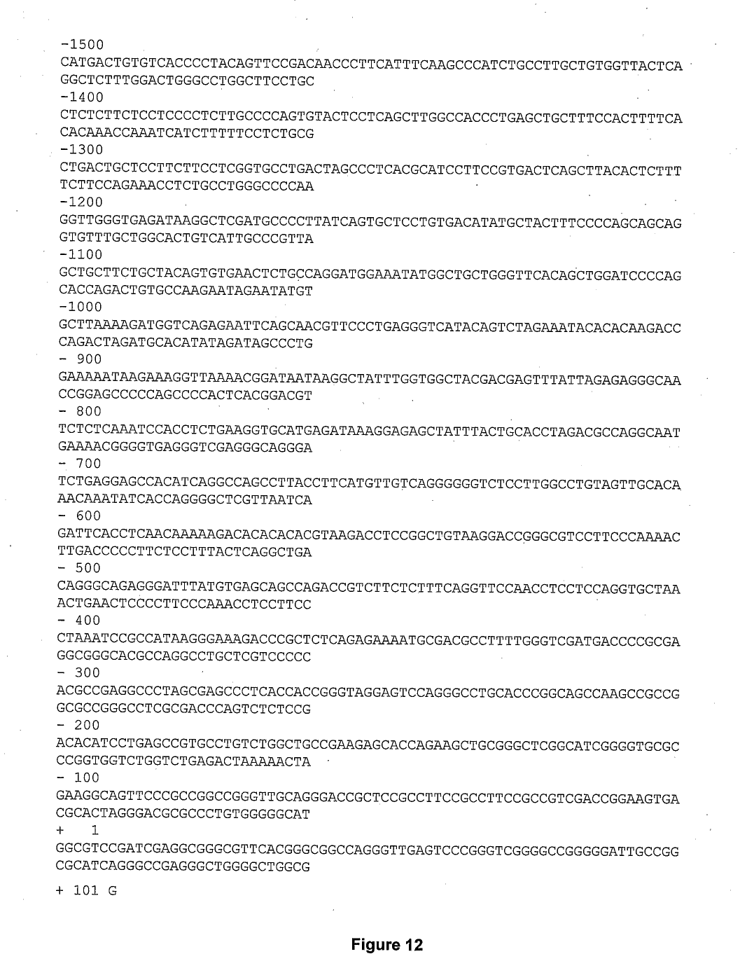

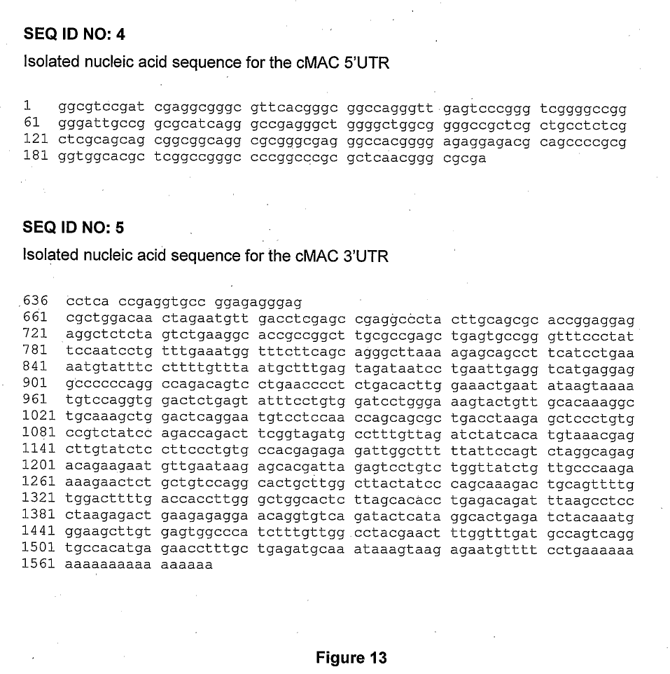

The invention also covers nucleic acids which are related to the transcription, function and stability of cMAC mRNA (Table 2, FIGS. 12 and 13):

TABLE 2

| Description | SEQ ID NO. |
|---|---|
| Isolated nucleic acid sequence for the cMAC genomic | SEQ ID NO. 3 |
| promoter region | |
| Isolated nucleic acid sequence for the cMAC 5′UTR | SEQ ID NO. 4 |
| Isolated nucleic acid sequence for the cMAC 3′UTR | SEQ ID NO. 5 |

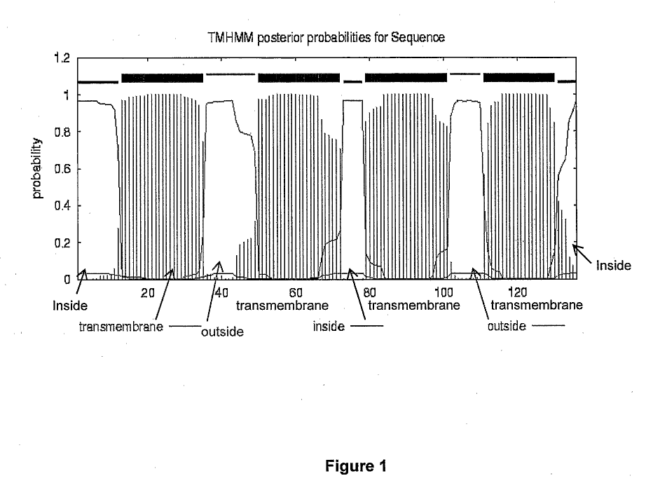

cMAC fragments of interest include, but are not limited to, those fragments containing amino acids of particular importance for normal cMAC function. Based on the predicted transmembrane structure of cMAC as illustrated in Table 3 and FIG. 1, the polypeptide can be subdivided into the following domains:

TABLE 3

| | Amino | | |
|---|---|---|---|
| | Acids | | |
| | (start, | Amino Acids | |
| Segment | end) | (standard symbols) | SEQ ID NO: |
| Inside | 1-12 | MLFSLRELVQWL | SEQ ID NO: 6 |
| Trans- | 13-35 | | |
| menbrane | | | |
| (TM) helix | | | |
| Outside | 36-49 | RVDGLVPGLSWWNV | SEQ ID NO: 7 |
| TM helix | 50-72 | | |
| Inside | 73-78 | QDGEKR | SEQ ID NO: 8 |
| TmHelix | 79-101 | | |
| Outside | 102-110 | CQKLAEQTR | SEQ ID NO: 9 |
| TM helix | 111-130 | | |
| Inside | 131-136 | RACRVN | SEQ ID NO: 10 |

The designation of inside and outside of the membrane needs to be experimentally confirmed however according to the analysis of the TMHMM prediction for human cMAC NM_{—}053045, NP_{—}444273.1, regions (e.g. epitopes) of particular interest for therapeutic antibodies include extra-membrane amino acid sequences 1-12, 36-49, 73-78, 102-110, 131-136.

Structure and Conservation of cMAC

The human cMAC cDNA encodes a hydrophobic protein of 136 amino acids. Analysis of the primary amino acid sequence with algorithms that predict transmembrane helices indicates that cMAC is an integral membrane protein with four transmembrane domains and short N- and C-terminal cytoplasmic domains FIG. 1. Two potential sites for post-translational modification of the protein were found using MotifsGCG programs. A potential protein kinase C (PKC) phosphorylation site and a Casein kinase II phosphorylation site were identified at Serine 4. The proposed PKC phosphorylation site at Ser 4 is predicted and is conserved in all the vertebrate cMAC genes identified. Additional potential PKC sites also exist at residue 73 (SVR) and 97 (SLK). The predominant PKC isoform present in T-cells is PKCθ, and it is known to be important in mediating T-cell activation. During T-cell activation PKCθ localizes to the plasma membrane lipid rafts (Khoshnan et al. J. Immunol. 165(12): 6933-40 (2000)), which are detergent insoluble cholesterol rich membrane domains containing many components (sometimes transiently upon stimulation) that contribute to the signal transduction. Interestingly, the proposed calcium channel in B-cells, CD20, also is a 4TM protein critical to B-cell activation. CD20 is known to be constitutively associated with lipid rafts. It remains to be determined if cMAC is a substrate for PKC but it is compelling to find a motif in cMAC amino acid sequence which may serve a regulatory function through PKC which is activated following TCR/CD28 stimulation. An isoprenylation site was also identified at cysteine 134 from the human, rat, mouse, zebrafish and Xenopus cMAC predicted proteins.

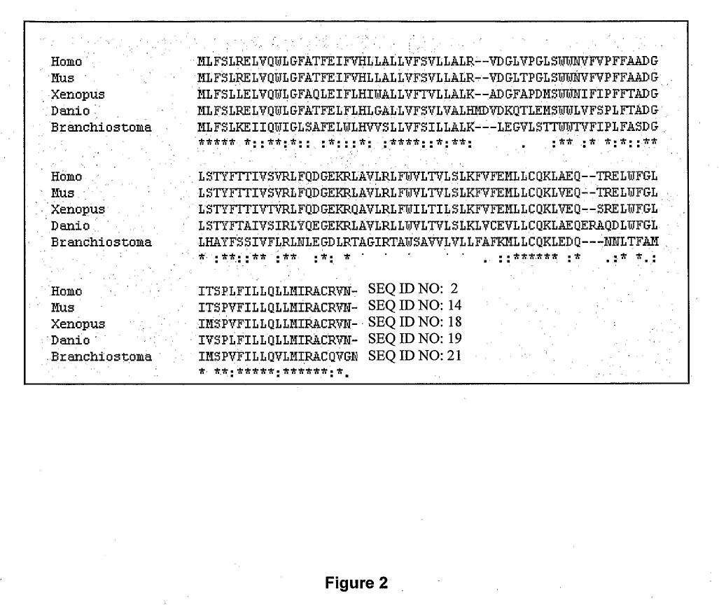

cMAC is a highly conserved protein. Orthologs of cMAC from other species disclosed here are set forth in FIGS. 2 and 15. The human cMAC protein is 97% identical to a predicted mouse protein, and 82% and 78% identical to Xenopus tropicalis and Danio rerio proteins, respectively. Interestingly there is also a gene encoded by the ancient vertebrate species Amphioxus floridae which is 54% identical to human cMAC. In total, 63 of the 136 amino acids are conserved across every vertebrate cMAC and 99/136 amino acids are identical or represent conservative changes. Interestingly, there are also similar proteins in invertebrates with Drosophila and C. elegans proteins of significantly lower levels of homologies (39% and 27% identical, respectively) compared with the vertebrate orthologs. It is not clear if the insect genes are indeed cMAC orthologs. The vertebrate and invertebrate proteins described here were mutually scoring best Blast hits suggesting they are likely orthologs and/or derived from a single ancestral gene It is interesting to note that cMAC protein sequence is most highly conserved in organisms containing a modern adaptive immune system. In particular, cMAC is highly conserved in animals containing T-cells (e.g. >80% identity in mammals, fish and amphibians), is more divergent in Amphioxus (54% identical), which has many orthologs and homologs of genes destined to be recruited for adaptive immunity but has not yet developed lymphocytes, and is most highly divergent in invertebrates which lack any correlate to lymphocytes. Thus, it is tempting to speculate that cMAC may have evolved along with the development of the vertebrate adaptive immune system and is a central player in capacitative calcium signaling required in T-cell activation and perhaps B-cell activation.

In each species, a single cMAC ortholog is present and conserved. cMAC is predicted to encode a 4 TM domain protein (illustration in FIG. 1). It is speculated that cMAC represents a novel gene family of calcium-mediated signal transducers.

Homologs and orthologs of cMAC include those disclosed herein (e.g. Table 4 and FIGS. 14 and 15), and those which would be apparent to one of skill in the art, and are meant to be included within the scope of the invention. For instance, screening assays for small molecules as contemplated in this invention could use a human cMAC homolog or a cMAC ortholog from a different species such as another primate, mammal, vertebrate or invertebrate. It is also contemplated that cMAC proteins include those isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

TABLE 4

| Description | SEQ ID NO. |
|---|---|
| cDNA sequence of mouse cMAC | SEQ ID NO. 11 |
| (GenBank Accession No. NM_177344). | |
| Mus musculus RIKEN cDNA C7300025P13 gene, | SEQ ID NO. 12 |
| mRNA (cDNA clone MGC: 31129 IMAGE: | |
| 4165766), complete cds. | |
| Full length amino acid sequence of mouse | SEQ ID NO. 13 |
| cMAC (translated from NM_177344.2) | |
| Mouse (mus musculus; full length | SEQ ID NO. 14 |
| amino acid sequence of mouse | |
| cMAC; >gi\|18490942\|gb\|AAH22606.1\| | |
| C730025P13Rik protein) | |
| Rat (Rattus norvegicus) cMAC | SEQ ID NO. 15 |
| (gi\|27706338\|ref\| | |
| XP_231050.1\|) | |
| Canis familiaris cMAC | SEQ ID NO. 16 |
| (>gi\|57092155\|ref\| | |
| XP_548356.1\|) | |
| Goat (Pan troglodytes) cMAC | SEQ ID NO. 17 |
| (>ref\|XP_520285.1\|: 114-249) | |
| Xenopus (Xenopus tropicalis) cMAC | SEQ ID NO. 18 |
| (>gi\|58332306\|ref\| | |
| NP_001011060.1\| | |
| hypothetical protein LOC496470) | |
| Zebra fish (Danio rerio) cMAC | SEQ ID NO. 19 |
| (>gi\|50540108\|ref\| | |
| NP_001002519.1\| | |
| hypothetical protein LOC436792) | |
| Chicken (Gallus gallus) cMAC | SEQ ID NO. 20 |
| (>gnl\|uniref100\| | |
| UniRef100_UPI00003AB3F7: | |
| 1-140 UPI00003AB3F7 UniRef100 entry) | |
| Mudfish (Branchiostoma floridae) cMAC | SEQ ID NO. 21 |
| >gnl\|uniref100\|UniRef100_Q71BB4 | |
| MGC14327-like protein | |

The present invention also includes any fragments of proteins or nucleic acids encoding fragments of proteins set forth in SEQ ID NOs: 1-21.

Additional homologs may be identified and readily isolated, without undue experimentation, by molecular biological techniques well known in the art. Further, there may exist genes at other genetic loci within the genome that encode proteins which have extensive homology to one or more domains of such gene products. These genes may also be identified via similar techniques.

Function and Role of cMAC, and Cell-Type Localization.

While not wishing to be bound to any particular theory, it is possible that human cMAC is a trans-membrane protein as illustrated in FIG. 1. Bioinformatic analysis indicates several domains likely to adopt an intra-membrane and extra-membrane conformation. This prediction highlights the potential use of antibodies to inhibit or activate cMAC in a therapeutic setting.

A function or biological activity of the cMAC polypeptide is clearly established in the functional activation of T-cells, as demonstrated by the Examples herein. Other biological activities of cMAC may be further elucidated as studies progress. Some of the more specific biological activities of cMAC, as such conclusions may be drawn based on the examples, include nuclear translocation of TORC, nuclear translocation of NFAT, and cAMP Response Element (CRE)-driven gene expression. cMAC could have several biochemical activities including as example as an ion channel, for example a calcium channel (voltage-gated or ligand gated); and may have activity in calcium dependent activation of a T-cell. Specific biochemical activities also include interactions with cell membranes and components of cell-membranes, as a target for myristilization, glycosylation, phosphorylation, de-phosphorylation and other post translational modifications. Based on the disclosure herein, those skilled in the art will be able to identify these and other biological activities of cMAC. The invention discloses a method of modulating (e.g. inhibiting or increasing) one or more of these activities of cMAC. Such methods may be for therapeutic application with identified modulators of cMAC, or for research and discovery use such as in screening assays or other research tools.

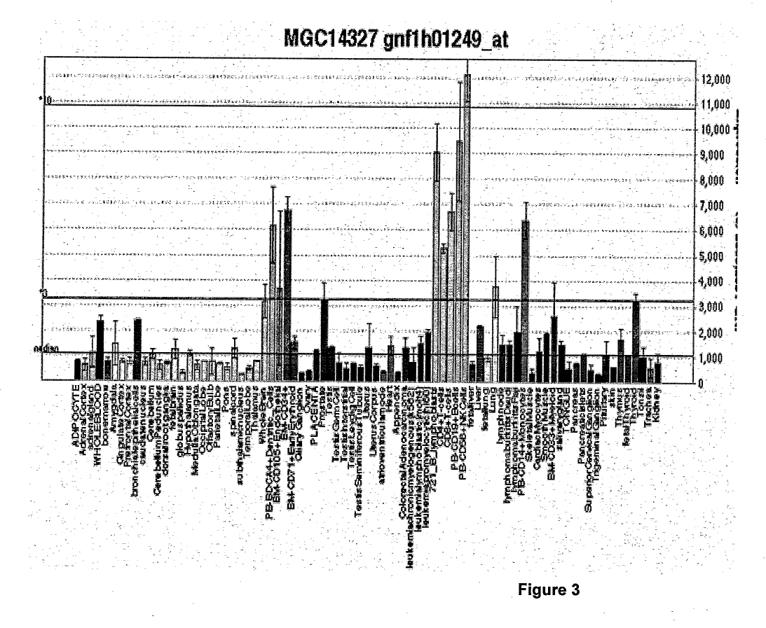

cMAC mRNA has been identified in a variety of human cell types. FIG. 3 identifies the highest level of cMAC in lymphocytes, specifically T-cells. Most other tissues also show some level of cMAC indicating that cMAC may be of general use in modulating disease related to calcium signaling.

Expression of cMAC. In order to gain an overview of cells expressing cMAC, the levels of mRNA in different tissues and cells types as indicated by Affymetrix expression profiling were examined. As shown in FIG. 3 cMAC was widely expressed. However, the highest levels of expression seen were in T and B-cell populations. The average expression level in these lymphocyte preparations were 3 to 10 fold higher than the median expression seen across all tissues examined. Although the expression of cMAC mRNA and protein will have to be examined by other methods, these results suggest that cMAC may be enriched in lymphocyte populations.

Predominant presence in T-cells indicates that anti-cMAC antibodies and other cMAC binding compounds will predominantly target T-cells; such antibodies or compounds have many significant therapeutic and research uses, as described more fully elsewhere in this specification.

Thus, the present invention provides isolated polypeptides comprising or consisting of an amino acid sequence as set forth in SEQ ID NO. 2 or a fragment thereof, or a substantially similar protein sequence having sequence identity of at least 50% with SEQ ID NO: 2, or a functional equivalent thereof exhibiting a biological activity selected from ion transport, ion diffusion, calcium dependent activation of a T-cell, nuclear translocation of TORC, nuclear translocation of NFAT or cAMP Response Element (CRE)-driven gene expression activity of native SEQ ID NO: 2. Accordingly, the present invention provides further the polypeptides of SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21. Further, such polypeptides may be, for example, a fusion protein including all or part of SEQ ID NO. 2.

The invention also includes isolated nucleic acid or nucleotide molecules, preferably DNA molecules, in particular SEQ ID NO. 1, SEQ ID NO: 11 or SEQ ID NO: 12 encoding the cMAC protein. The invention also discloses an isolated nucleic acid molecule, preferably a DNA molecule, of the present invention encoding a polypeptide comprising the amino acid sequence set forth in SEQ ID NO:2 or SEQ ID NO:13 to 21.

The invention also encompasses: (a) vectors that comprise a nucleotide sequence of a cMAC protein, particularly SEQ ID NO. 1, SEQ ID NO: 11 or SEQ ID NO: 12 or a fragment thereof and/or their complements (i.e., antisense); (b) vector molecules, preferably vector molecules comprising transcriptional control sequences, in particular expression vectors, which comprise coding sequences of any of the cMAC proteins disclosed herein operatively associated with a regulatory element that directs the expression of the coding sequences; and (c) genetically engineered host cells that contain a vector molecule as mentioned herein or at least a fragment of any of the foregoing nucleotide sequences operatively associated with a regulatory element that directs the expression of the coding sequences in the host cell. As used herein, regulatory elements include, but are not limited to, inducible and non-inducible promoters, enhancers, operators and other elements known to those skilled in the art that drive and regulate expression. Preferably, host cells can be vertebrate host cells, preferably mammalian host cells, such as human cells or rodent cells, such as CHO or BHK cells. Likewise preferred, host cells can be bacterial host cells, in particular E. coli cells.

The invention therefore covers a vector molecule comprising the nucleic acid sequence of cMAC (SEQ ID NO. 1), and a host cell comprising such vector molecule.

Particularly preferred is a host cell, in particular of the above described type, which can be propagated in vitro and which is capable upon growth in culture of producing a cMAC polypeptide, in particular a polypeptide comprising or consisting of an amino acid sequence set forth in SEQ ID NO: 2, wherein said cell comprises at least one transcriptional control sequence that is not a transcriptional control sequence of the natural endogenous human gene encoding said polypeptide, wherein said one or more transcriptional control sequences control transcription of a DNA encoding said polypeptides.

This vector or host cell may be used in a method for producing a cMAC polypeptide of SEQ ID NO. 2 comprising culturing a host cell having incorporated therein an expression vector comprising the cMAC vector under conditions sufficient for expression of the polypeptide in the host cell.

The invention also includes fragments of any of the nucleic acid sequences disclosed herein. Fragments of the nucleic acid sequences of SEQ ID NO. 1 and SEQ ID NO. 3 may be used as a hybridization probe for a cDNA library to isolate the full length gene and to isolate other genes which have a high sequence similarity to a cMAC gene of similar biological activity. Probes of this type preferably have at least about 20 bases and may contain, for example, from about 30 to about 50 bases, about 50 to about 100 bases, about 100 to about 200 bases, or more than 200 bases. The probe may also be used to identify a cDNA clone corresponding to a full length transcript and a genomic clone or clones that contain a complete cMAC gene including regulatory and promoter regions, exons, and introns. An example of a screen comprises isolating the coding region of a cMAC gene by using the known DNA sequence to synthesize an oligonucleotide probe. Labeled oligonucleotides having a sequence complementary to that of the gene of the present invention are used to screen a library of human cDNA, genomic DNA or mRNA to determine to which members of the library the probe hybridizes.

The isolated nucleotide sequence of the present invention encoding a cMAC polypeptide may be labeled and used to screen a cDNA library constructed from mRNA obtained from the organism of interest. Hybridization conditions will be of a lower stringency when the cDNA library was derived from an organism different from the type of organism from which the labeled sequence was derived. Alternatively, the labeled fragment may be used to screen a genomic library derived from the organism of interest, again, using appropriately stringent conditions. Such low stringency conditions will be well known to those of skill in the art, and will vary predictably depending on the specific organisms from which the library and the labeled sequences are derived. For guidance regarding such conditions see, for example, Sambrook et al. cited above.

PCR technology may also be utilized to isolate full or partial cDNA sequences which are substantially similar to cMAC. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, supra.

In cases where the gene identified is the normal, or wild type, gene, this gene may be used to isolate mutant alleles of the gene. Such an isolation is preferable in processes and disorders which are known or suspected to have a genetic basis. Mutant alleles may be isolated from individuals either known or suspected to have a genotype which contributes to disease symptoms related to inflammation or immune response. Mutant alleles and mutant allele products may then be utilized in the diagnostic assay systems described below.

A cDNA of the mutant gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probed to identify the corresponding mutant allele in the library. The clone containing this gene may then be purified through methods routinely practiced in the art, and subjected to sequence analysis as described above.

The present invention includes proteins that represent functionally equivalent cMAC gene products. Such an equivalent gene product may contain deletions, additions or substitutions of amino acid residues within the amino acid sequence encoded by the gene sequences described, above, thus producing a functionally equivalent gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Data disclosed herein indicate particular polypeptide fragments are useful to certain activities of the cMAC protein. Thus, these cMAC peptide fragments as well as fragments of the nucleic acids encoding the active portion of the cMAC polypeptides disclosed herein, and vectors comprising said fragments, are also within the scope of the present invention. As used herein, a fragment of the nucleic acid encoding the active portion of the cMAC polypeptides refers to a nucleotide sequence having fewer nucleotides than the nucleotide sequence encoding the entire amino acid sequence of a cMAC polypeptide and which encodes a peptide having an activity of a cMAC protein (i.e., a peptide having at least one biological activity of a cMAC protein) as defined herein. Generally, the nucleic acid encoding a peptide having an activity of a cMAC protein will be selected from the bases encoding the mature protein. However, in some instances, it may be desirable to select all or part of a peptide from the leader sequence portion of the nucleic acids of a cMAC protein. These nucleic acids may also contain linker sequences, modified restriction endonuclease sites and other sequences useful for molecular cloning, expression or purification or recombinant peptides having at least one biological activity of a cMAC protein. cMAC peptide fragments as well as nucleic acids encoding a peptide fragment having an activity of a cMAC protein may be obtained according to conventional methods.

In addition, antibodies directed to these peptide fragments may be made as described herein below. Modifications to these polypeptide fragments (e.g., amino acid substitutions) which may increase the immunogenicity of the peptide, may also be employed. Similarly, using methods familiar to one of skill in the art, said peptides of the cMAC proteins may be modified to contain signal or leader sequences or conjugated to a linker or other sequence to facilitate molecular manipulations.

The polypeptides of the present invention may be produced by recombinant DNA technology using techniques well known in the art. Therefore, there is provided a method of producing a polypeptide of the present invention, which method comprises culturing a host cell having incorporated therein an expression vector containing an exogenously-derived polynucleotide encoding a polypeptide comprising an amino acid sequence as set forth in SEQ ID NOs: 2, 13-21, preferably SEQ ID NO. 2, under conditions sufficient for expression of the polypeptide in the host cell, thereby causing the production of the expressed polypeptide. Optionally, said method further comprises recovering the polypeptide produced by said cell. In a preferred embodiment of such a method, said exogenously-derived polynucleotide encodes a polypeptide consisting of an amino acid sequence set forth in SEQ ID NO: 2. Preferably, said exogenously-derived polynucleotide comprises the nucleotide sequence as set forth in any of SEQ ID NOs: 1.

Thus, methods for preparing the polypeptides and peptides of the invention by expressing nucleic acid encoding respective polypeptide sequences are described herein. Methods that are well known to those skilled in the art can be used to construct expression vectors containing protein-coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, supra, and Ausubel et al., 1989, supra. Alternatively, RNA capable of encoding differentially expressed gene protein sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

A variety of host-expression vector systems may be utilized to express the gene coding sequences of the invention. Such host-expression systems represent vehicles by which the coding sequences of interest may be produced and subsequently purified, but also represent cells which may, when transformed or transfected with the appropriate nucleotide coding sequences, exhibit the protein of the invention in situ. These include but are not limited to microorganisms such as bacteria (e.g., E. coli, B. subtilis) transformed with recombinant bacteriophage DNA, plasmid DNA or cosmid DNA expression vectors containing the cMAC protein coding sequences; yeast (e.g. Saccharomyces, Pichia) transformed with recombinant yeast expression vectors containing cMAC protein coding sequences; insect cell systems infected or transfected with recombinant virus expression vectors (e.g., baculovirus) containing cMAC protein coding sequences; plant cell systems infected with recombinant virus expression vectors (e.g., cauliflower mosaic virus, CaMV; tobacco mosaic virus, TMV) or transformed with recombinant vectors, including plasmids, (e.g., Ti plasmid) containing cMAC protein coding sequences; or mammalian cell systems (e.g. COS, CHO, BHK, 293, 3T3) harboring recombinant expression constructs containing promoters derived from the genome of mammalian cells (e.g., metallothioneine promoter) or from mammalian viruses (e.g., the adenovirus late promoter; the vaccinia virus 7.5K promoter, or the CMV promoter).

Expression of the cMAC proteins of the present invention by a cell from a cMAC-encoding gene that is native to the cell can also be performed. Methods for such expression are detailed in, e.g., U.S. Pat. Nos. 5,641,670; 5,733,761; 5,968,502; and 5,994,127, all of which are incorporated by reference herein in their entirety. Cells that have been induced to express cMAC by the methods of any of U.S. Pat. Nos. 5,641,670; 5,733,761; 5,968,502; and 5,994,127 can be implanted into a desired tissue in a living animal in order to increase the local concentration of cMAC in the tissue. Such methods have therapeutic implications for, e.g., neurodegenerative conditions in which loss of CREB function occurs and as such agonists and/or exogenous cMAC protein may be useful to treat said conditions.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable. In this respect, fusion proteins comprising hexahistidine tags may be used (Sisk et alk, 1994: J. Virol 68: 766-775) as provided by a number of vendors (e.g. Qiagen, Valencia, Calif.). Such vectors include, but are not limited, to the E. coli expression vector pUR278 (Ruther et al., 1983, EMBO J. 2:1791), in which the protein-encoding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

Promoter regions can be selected from any desired gene using vectors that contain a reporter transcription unit lacking a promoter region, such as a chloramphenicol acetyl transferase (“CAT”), or the luciferase transcription unit, downstream of restriction site or sites for introducing a candidate promoter fragment; i.e., a fragment that may contain a promoter. For example, introduction into the vector of a promoter-containing fragment at the restriction site upstream of the CAT gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well-known and readily available promoters, but also promoters that readily may be obtained by the foregoing technique, using a reporter gene.

Among known bacterial promoters suitable for expression of polynucleotides and polypeptides in accordance with the present invention are the E. coli lacI and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

In an insect system, Autographa californica nuclear polyhedrosis virus (AcNPV) is one of several insect systems that can be used as a vector to express foreign genes. The virus grows in Spodoptera frugiperda cells. The coding sequence may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the desired protein in infected hosts. (E.g., See Logan & Shenk, 1984, Proc. Natl. Acad. Sci. USA 81:3655-3659). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544). Other common systems are based on SV40, retrovirus or adeno-associated virus. Selection of appropriate vectors and promoters for expression in a host cell is a well-known procedure and the requisite techniques for expression vector construction, introduction of the vector into the host and expression in the host per se are routine skills in the art. Generally, recombinant expression vectors will include origins of replication, a promoter derived from a highly expressed gene to direct transcription of a downstream structural sequence, and a selectable marker to permit isolation of vector containing cells after exposure to the vector.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

The present invention also includes recombinant cMAC peptides and peptide fragments having an activity of a cMAC protein. The term “recombinant peptide” refers to a protein of the present invention which is produced by recombinant techniques, wherein generally DNA encoding a cMAC active fragment is inserted into a suitable expression vector which is in turn used to transform a host cell to produce the heterologous protein.

Recombinant proteins of the present invention also may include chimeric or fusion proteins of cMAC and different polypeptides which may be made according to techniques familiar to one of skill in the art (see, for example, Current Protocols in Molecular Biology; Eds Ausubel et al. John Wiley & Sons; 1992; PNAS 85:4879 (1988)).

For long-term, high-yield production of recombinant proteins, stable expression is preferred. For example, cell lines which stably express the differentially expressed gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the differentially expressed gene protein. Such engineered cell lines may be particularly useful in screening and evaluation of compounds that affect the endogenous activity of the expressed protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

An alternative fusion protein system allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described below, a protein of the present invention may be labeled, either directly or indirectly, to facilitate detection of a complex formed between the protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels.

Where recombinant DNA technology is used to produce a protein of the present invention for such assay systems, it may be advantageous to engineer fusion proteins that can facilitate labeling, immobilization, detection and/or isolation.

Indirect labeling involves the use of a protein, such as a labeled antibody, which specifically binds to a polypeptide of the present invention. Such antibodies include but are not limited to polyclonal, monoclonal, chimeric, single chain, Fab fragments and fragments produced by a Fab expression library.

Use of cMAC as Drug Target, in Screening Assays, and for Identification of cMAC Modulators

The instant invention discloses for the first time that cMAC is a useful drug target for therapeutic agents for the treatment of pathological conditions related to abnormal activation of T-cells. The disclosure establishes that modulators (e.g. agonists or inhibitors) of cMAC activity and/or expression may have many significant therapeutic uses. Pathological conditions that may be treated with modulators of cMAC include, but are not limited to, cMAC-associated disorders (as defined above).

### Screening

In yet another aspect, the present invention relates to a method to identify modulators useful to treat the pathological conditions discussed above comprising assaying for the ability of a candidate modulator to inhibit or enhance cMAC activity and/or inhibit or enhance cMAC expression in vitro, ex vivo or in vivo.

Based on the instant disclosure, conventional screening assays (e.g., in vitro, ex vivo and in vivo) may be used to identify modulators that inhibit or enhance cMAC protein activity and/or inhibit or enhance cMAC expression.

Many formats for such assays are available and known to those skilled in the art. Broadly speaking such assays are based on radiolabel, fluorescence, luminescence, substrate accumulation and a wide range of other basic formats. Assays can be designed to employ the target protein in a purified, partially purified, cell extract, whole cell or multi-cell format. Assays can generally be designed as high-throughput or low-throughput. The target protein activity may be measured directly or indirectly.

The activity of cMAC that could be measured in an assay includes any activity such as a function or biological activity of the cMAC polypeptide established in the instant disclosure, including the functional activation of T-cells. Other biological activities of cMAC may be enhanced or inhibited in a screening assay include nuclear translocation of TORC, nuclear translocation of NFAT or increased expression of NFAT dependent transcribed genes markers or reporters, and cAMP Response Element (CRE)-driven gene expression, markers of T-cell activation such as ICOS, CD69, CD40L and CD25. cMAC also may function as an ion channel, for example a calcium channel (voltage-gated or ligand-gated); and may have activity in calcium dependent activation of a T-cell. Thus cMAC activity could be monitored by effects on ion influx or efflux in cell culture. At a more specific biochemical level, biological activities that could be assayed also include interactions with cell membranes and components of cell-membranes, as a target for myristilization, glycosylation, phosphorylation, de-phosphorylation and other post translational modifications. Based on the disclosure herein, those skilled in the art will be able to identify these and other biological activities of cMAC, any of which could give rise to a suitable screening assay.

Such assays typically employ controls, such as negative and/or positive controls which establish the background activity of cMAC. Potential agents, such as small molecules, antibodies or antibody fragments, and the like, are tested sequentially in the assay to identify those agents which generate a measurable effect on the activity of interest when compared to the controls. Those skilled in the art are familiar with the testing of agents, particularly large libraries of agents, in screening formats which may be high-throughput or low-throughput.

The invention therefore comprises:

A method for identifying a compound useful for the treatment of a cMAC-associated disorder comprising contacting a test compound with cMAC; and detecting a change of a biological activity of cMAC compared to cMAC not contacted with the test compound, wherein detecting a change identifies said test compound as useful for the treatment of said disorder.

A method of identifying a compound useful for treatment of a cMAC-associated disorder comprising contacting a test compound with cMAC under sample conditions permissive for cMAC biological activity determining the level of a cMAC biological activity in vitro or in vivo; comparing said level to that of a control sample lacking said test compound; and, selecting a test compound which causes said level to change for further testing as a potential agent for treatment of said disorder; and a method for testing if a compound modulates a cMAC biological activity comprising: contacting in vitro or in vivo a test compound with cMAC; and detecting a change of a biological activity of cMAC compared to cMAC not contacted with the test compound, wherein detecting a change identifies said test compound as a modulator of cMAC biological activity.

In one embodiment the method identifies inhibitors of cMAC biological activity. The biological activity may be selected from among ion transport, ion diffusion, protein-cMAC interaction or cMAC modification, calcium dependent activation of a T-cell, nuclear translocation of TORC or NFAT or another calcium dependent molecule, calcineurin pathway activation, and cAMP Response Element (CRE)- or NFAT driven gene expression.

The invention includes further identifying and confirming a compound is useful for the treatment of a cMAC-associated disorder comprising administering a compound identified in an in vitro screening assay to an animal model of said cMAC-associated disorder and observing a desired response in said animal.

As contemplated herein, the instant invention includes a method to use the cMAC gene and gene product disclosed herein to discover agonists and antagonists that induce or repress, respectively, TORC activity, NFAT (nuclear factor of activated T-cells) activation, and/or T-cell activation, and result in various therapeutic effects.

In further embodiments, the invention relates a method for identifying a compound useful for the treatment of a cMAC-related disorder comprising (a) contacting a test compound with cMAC; and (b) detecting a change of a biological activity of cMAC compared to cMAC not contacted with the test compound, wherein detecting a change identifies said test compound as useful for the treatment of said disorder.

The invention includes a method of identifying a compound useful for treatment of a cMAC-related disorder comprising (a) contacting a test compound with cMAC under sample conditions permissive for cMAC biological activity; (b) determining the level of a cMAC biological activity; (c) comparing said level to that of a control sample lacking said test compound; and, (d) selecting a test compound which causes said level to change for further testing as a potential agent for treatment of said disorder. Alternatively, the invention relates to a method for testing if a compound modulates a cMAC biological activity comprising (a) contacting a test compound with cMAC; and (b) detecting a change of a biological activity of cMAC compared to cMAC not contacted with the test compound, wherein detecting a change identifies said test compound as a modulator of cMAC biological activity.

As related elsewhere herein, the change to be identified may be a reduction of a biological activity, such as a reduction of ion transport, ion diffusion, protein-cMAC interaction or cMAC modification, calcium dependent activation of a T-cell, activation of a T-cell, or markers of T-cell activation including but not limited to (ICOS, CD69, CD25, CD40L), nuclear translocation of NFAT or TORC, cAMP Response Element (CRE)-driven gene expression and NFAT or TORC driven gene expression.

The invention further comprises the use of any compound identified by a screening assay method herein in the treatment of a cMAC-associated disorder.

The invention includes a method to identify modulators useful to treat a disorder comprising assaying for the ability of a candidate modulator to enhance or inhibit the expression of a cMAC protein.

Numerous formats for such assays are known to those skilled in the art. Inhibitors of cMAC expression can be identified by testing candidate modulators for their ability to inhibit cMAC mRNA transcription, processing, export to the cytosol, stability, translation (or any of the numerous sub-steps involved in these processes). Ultimately inhibitors of expression are identified by a reduction in the amount of functionally active cMAC protein compared to controls. Enhancers of expression can enhance any of the steps leading to expression and ultimately result in an increase in the amount of functionally active cMAC protein.

Typical expression assays include promoter activity assays. In a standard promoter assay, a vector is constructed comprising all or part of the cMAC promoter sequence of SEQ ID NO. 3 operably linked to a reporter gene sequence (encoding a reporter protein) such as CAT (Chloramphenicol acetyl-transferase) or luciferase. Especially preferred is the promoter sequence of SEQ ID NO. 3 that does not include sequences corresponding to the cDNA sequence of SEQ ID NO. 1. The vector is transfected into a cell or cell extract. Candidate modulators are tested to determine if they increase promoter activity by measuring the activity of the reporter protein compared to controls. Numerous other formats of promoter activity assays are known and available to those skilled in the art.

cMAC gene expression (e.g. mRNA levels) may also be determined using methods familiar to one of skill in the art, including, for example, conventional Northern analysis or commercially available microarrays. Additionally, the effect of a test compound on cMAC levels and/or related regulatory protein levels can be detected with an ELISA antibody-based assay or fluorescent labeling reaction assay. These techniques are readily available for high throughput screening and are familiar to one skilled in the art.

The promoter fragment can also be readily inserted into any promoter-less reporter gene vector designed for expression in human cells (e.g. Clontech promoter-less enhanced fluorescent protein vector pECFP-1, pEGFP-1, or pEYFP, Clontech, Palo Alto, Calif.). The screen would then consist of culturing the cells for an appropriate length of time with a different compound added to each well and then assaying for reporter gene activity.

In another embodiment, an assay for modulators of cMAC expression comprises first screening cell lines to find ones that express the cMAC protein of interest. Recombinant cell lines containing an expressing an exogenous cMAC gene can also be tested. These cell lines could be cultured in, for example, 96, 384 or 1536 well plates. A comparison of the effects of some known modifiers of gene expression e.g. dexamethasone, phorbol ester, heat shock on primary tissue cultures and the cell lines will allow the selection of the most appropriate cell line to use. The screen would then merely consist of culturing the cells for a set length of time with a different compound added to each well and then assaying for cMAC activity.

Data gathered from these studies may be used to identify those modulators with therapeutic usefulness for the treatment of the pathological conditions discussed above; e.g. inhibitory substances could be further assayed in conventional in vitro or in vivo models of said pathological conditions and/or in clinical trials with humans with said pathological conditions according to conventional methods to assess the ability of said compounds to treat said pathological conditions in vivo.

The present invention, by making available critical information regarding the active portions of cMAC polypeptides, allows the development of modulators of cMAC function e.g., antibodies, antibody fragments, small molecule agonists or antagonists, by employing rational drug design familiar to one of skill in the art.

Use of cMAC Modulators in the Treatment of cMAC-Associated Disorders

In another aspect, the invention relates to a method to treat cMAC-associated disorders comprising administering to a subject in need thereof a pharmaceutical composition comprising an effective amount of a cMAC modulator.

It is contemplated that modulators identified and discovered through the cMAC screening assays disclosed herein could be agents such as small molecules, including organic small molecules (with or without drug-like features), and including natural products. Modulators of cMAC include agonists of cMAC biological activity or cMAC expression; Modulators also include inhibitors of cMAC biological activity or inhibitors of cMAC expression. Further details are provided elsewhere herein.

Use of cMAC Modulators to Modulate Biological Processes

The invention discloses methods of inhibiting biological processes. In one embodiment, the invention relates to a method of inhibiting cMAC biological activity in a cell. Such inhibition may be achieved by contacting a cell with an inhibitor of cMAC, such as an anti-cMAC antibody, antibody fragment, or polypeptide comprising a cMAC-specific binding region or with a nucleic acid which reduces cMAC expression. The biological activity which is inhibited is selected from among the group consisting of calcium dependent activation of a T-cell, nuclear translocation of TORC, cAMP Response Element (CRE)-driven gene expression, and expression of NFAT inducible genes/proteins such as IL-2.

Alternatively, the biological activity may be a method of selectively inhibiting lymphocyte activity in a multi-cellular organism comprising contacting said organism with an anti-cMAC antibody, antibody fragment, or polypeptide comprising a cMAC-specific binding region or with a nucleic acid which reduces cMAC expression. ‘Selectively’ as used herein means tending to select the identified tissue or cell type in preference to other tissue or cell types

As for methods of enhancing biological processes, the invention also relates to a method of enhancing T-cell activation comprising contacting a T-cell or a T-cell precursor cell with a purified cMAC polypeptide, a gene therapy vector comprising the cMAC gene, or an enhancer of cMAC gene expression.

Further details on the use of cMAC modulators to modulate biological processes are provided elsewhere herein.

Antibodies to cMAC

Suitable antibodies to cMAC proteins can be produced according to conventional methods. For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above, all as described in the definition of ‘antibody’, supra.

For the production of antibodies to the cMAC polypeptides discussed herein, various host animals may be immunized by injection with the polypeptides, or a portion thereof. Such host animals may include, but are not limited to, rabbits, mice, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Antibodies that bind the cMAC polypeptides disclosed herein can be prepared using full length cMAC polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are) formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes of cMAC may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

A wide variety of antibody/immunoglobulin frameworks or scaffolds can be employed so long as the resulting polypeptide includes one or more binding region which is specific for the cMAC protein. Such frameworks or scaffolds include the 5 main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

Alternatively, known or future non-immunoglobulin frameworks and scaffolds may be employed, as long as they comprise a binding region specific for the cMAC protein of SEQ ID NO: 2. Such compounds are known herein as “polypeptides comprising a cMAC-specific binding region”. Known non-immunoglobulin frameworks or scaffolds include Adnectins (fibronectin) (Compound Therapeutics, Inc., Waltham, Mass.), ankyrin (Molecular Partners AG, Zurich, Switzerland), domain antibodies (Domantis, Ltd (Cambridge, Mass.) and Ablynx nv (Zwijnaarde, Belgium)), lipocalin (Anticalin) (Pieris Proteolab AG, Freising, Germany), small modular immuno-pharmaceuticals (Trubion Pharmaceuticals Inc., Seattle, Wash.), maxybodies (Avidia, Inc. (Mountain View, Calif.)), Protein A (Affibody AG, Sweden) and affilin (gamma-crystallin or ubiquitin) (Scil Proteins GmbH, Halle, Germany).

According to the instant invention, the anti-cMAC antibody or fragment thereof, or the polypeptide comprising a cMAC-specific binding region, regardless of the framework or scaffold employed, may be bound, either covalently or non-covalently, to an additional moiety. The additional moiety may be a polypeptide, an inert polymer such as PEG, small molecule, radioisotope, metal, ion, nucleic acid or other type of biologically relevant molecule. Such a construct, which may be known as an immunoconjugate, immunotoxin, or the like, is also included in the meaning of antibody, antibody fragment or polypeptide comprising a cMAC-specific binding region, as used herein.

The invention further relates to the use of an antibody, an antibody fragment specific for cMAC or a polypeptide comprising a cMAC-specific binding region in the treatment of a disorder in a subject as described herein.

The invention also relates to an antibody or antibody fragment which binds specifically to cMAC (SEQ ID NO. 2) or a polypeptide comprising a cMAC-specific binding region, including an antibody fragment (e.g. Fab or F(ab′)2 fragment) or a monoclonal antibody. The invention also covers a pharmaceutical composition of such antibody, antibody fragment or binding region containing polypeptide which binds specifically to cMAC.

Detection of cMAC by the antibodies described herein may be achieved using standard ELISA, FACS analysis, and standard imaging techniques used in vitro or in vivo. Detection can be facilitated by coupling (i.e., physically linking) cMAC to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

Particularly preferred, for ease of detection, is the sandwich assay, of which a number of variations exist, all of which are intended to be encompassed by the present invention. For example, in a typical forward assay, unlabeled anti-cMAC antibody is immobilized on a solid substrate and the sample to be tested brought into contact with the bound molecule. After a suitable period of incubation, for a period of time sufficient to allow formation of an antibody-antigen binary complex, a second antibody, labeled with a reporter molecule capable of inducing a detectable signal, is added and incubated, allowing time sufficient for the formation of a ternary complex of antibody-antigen-labeled antibody. Any unreacted material is then washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the cMAC polypeptides or related regulatory proteins, or fragments thereof.

The most commonly used reporter molecules are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The invention therefore includes pharmaceutical compositions comprising antibodies that are highly selective for human cMAC or portions of human cMAC polypeptides, and methods of using such antibodies. Upon administration to a subject, such antibodies may inhibit or decrease cMAC activity, or in some cases may increase cMAC activity, by interacting directly with the protein. Inhibitors may block active sites or block access of substrates to active sites. cMAC antibodies may also be used to inhibit cMAC activity by preventing protein-protein interactions that may be involved in the regulation of cMAC proteins and necessary for protein activity. Antibodies with inhibitory activity such as described herein can be produced and identified according to standard assays familiar to one of skill in the art.

cMAC antibodies may also be used diagnostically. For example, one could use these antibodies according to conventional methods to quantitate levels of a cMAC protein in a subject; increased levels could, for example, indicate undesirable T-cell activation, excessive activation of CRE-dependent gene expression (e.g. activation of genes that have CRE in their promoter regions) and could possibly indicate the degree of excessive activation and corresponding severity of related pathological condition. Thus, different cMAC levels could be indicative of various clinical forms or severity of pathological conditions such as cMAC-associated disorders. Such information would also be useful to identify subsets of patients suffering from a pathological condition that may or may not respond to treatment with cMAC modulators.

### Gene Therapy

In another embodiment, nucleic acids comprising a sequence encoding a cMAC protein or functional derivative thereof are administered for therapeutic purposes, by way of gene therapy. Gene therapy refers to therapy performed by the administration of a nucleic acid to a subject. In this embodiment of the invention, the nucleic acid produces its encoded protein that mediates a therapeutic effect by promoting normal T-cell activation.

Any of the methods for gene therapy available in the art can be used according to the present invention. Exemplary methods are described below.

In a preferred aspect, the therapeutic comprises a cMAC nucleic acid that is part of an expression vector that expresses a cMAC protein or fragment or chimeric protein thereof in a suitable host. In particular, such a nucleic acid has a promoter operably linked to the cMAC coding region, said promoter being inducible or constitutive, and, optionally, tissue-specific. In another particular embodiment, a nucleic acid molecule is used in which the cMAC coding sequences and any other desired sequences are flanked by regions that promote homologous recombination at a desired site in the genome, thus providing for intrachromosomal expression of a cMAC nucleic acid (Koller and Smithies, 1989, Proc. Natl. Acad. Sci. USA 86:8932-8935; Zijistra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; and WO 93/20221). Alternatively, the nucleic acid can be introduced intracellularly and incorporated within host cell DNA for expression, by homologous recombination (see, e.g., U.S. Pat. Nos. 5,413,923; 5,416,260; and 5,574,205; and Zijlstra et al., 1989, Nature 342:435-438).

In a specific embodiment, a viral vector that contains a cMAC nucleic acid is used. For example, a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The cMAC nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety.

Adeno-associated virus (MV) has also been proposed for use in gene therapy. Methods for producing and utilizing MV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a cMAC nucleic acid is introduced into the cells such that it is expressible by the cells or their progeny, and the recombinant cells are then administered in vivo for therapeutic effect. In a specific embodiment, stem or progenitor cells are used. Any stem—and/or progenitor cells that can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (see, e.g., WO 94/08598), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique that provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The pharmaceutical compositions of the present invention may also comprise substances that inhibit the expression of cMAC proteins at the nucleic acid level. Such molecules include ribozymes, antisense oligonucleotides, triple helix DNA, RNA aptamers, siRNA, and double or single stranded RNA directed to an appropriate nucleotide sequence of a cMAC nucleic acid. These inhibitory molecules may be created using conventional techniques by one of skill in the art without undue burden or experimentation. For example, modifications (e.g. inhibition) of gene expression can be obtained by designing antisense molecules, DNA or RNA, to the control regions of a gene encoding a cMAC polypeptide discussed herein, i.e. to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences, for example, the mRNA for cMAC.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using RNA interference (“RNAi”) strategies. RNAi is a relatively new discovery. It relies on double stranded RNA. A description of such technology may be found in WO 99/32619 which is hereby incorporated by reference in its entirety. RNAi technology has proven useful as a means to inhibit gene expression (see for example, Cullen, B R Nat. Immunol. 2002 July; 3(7):597-9).

An RNAi agent as used herein refers to compounds and compositions which can act through an RNAi mechanism (see, for general reference, He and Hannon, (2004) Nat. Genet. 5:522-532). RNAi agents such as short interfering RNA (“siRNA”), double stranded RNA (“dsRNA”), short hairpin RNA (“shRNA”, also sometimes called ‘synthetic RNA’) are commonly used, others are in development. When introduced into a cell or synthesized within a cell RNAi agents are incorporated into a macromolecular complex which uses strands of the RNAi agent to target and cleave RNA strands containing the complementary (or substantially complementary) sequence.

RNAi agents may be chemically modified. A variety of suitable chemical modifications known to those skilled in the art are set forth in PCT publication WO 03/070918, incorporated herein by reference. Other modifications and combinations of modifications that do not abolish the RNAi activity of the compound are also contemplated herein.

RNAi agents suitable for use in the invention include the dsRNA strands resulting from the hybridization of the single stranded sense and antisense strands indicated in Table 5, and Table 6 (see Examples) (see Examples; note that sequences must are synthesized as RNA (not DNA), and may optionally be chemically modified).

The RNAi agents that can be prepared based on Table 5 or Table 6, or as otherwise designed by one skilled in the art, can be shortened to 17 to 30 mer double stranded compounds, with or without 3′ overhangs of 1-6 nts, with or without chemical modifications or end modifications, and with or without exact complementarity to the target sequence, in which case they are referred to here in as short interfering RNA (“siRNA”) compounds.

Preferred siRNA compounds, as calculated using the Biopred algorithm (Huesken et al. (2005) Nat. Biotech. 23(8):995-1001) are:

TABLE 5

| | | | |
|---|---|---|---|
| | SEQ ID | | SEQ ID |
| siRNA guide sequence (5′ -> 3′) | NO.: | siRNA complement (5′ -> 3′) | NO.: |

| | | | |
|---|---|---|---|
| UAGUAAGCCAAGCAGUGCCTG | 22 | GGCACUGCUUGGCUUACUATT | 62 |
| UUGUGCAACAGUACUUUCCCA | 23 | GGAAAGUACUGUUGCACAATT | 63 |
| UUACUUAUAUUCAGUUUCCAA | 24 | GGAAACUGAAUAUAAGUAATT | 64 |
| UAUGAGUAUCUGACACCUGTT | 25 | CAGGUGUCAGAUACUCAUATT | 65 |
| UAAGAGUGCCAGCCCAAGGTG | 26 | CCUUGGGCUGGCACUCUUATT | 66 |
| UAGUUGACCCGACAGGCGCGG | 27 | GCGCCUGUCGGGUCAACUATT | 67 |
| UCGUAGGCCAACAAAGAUGGG | 28 | CAUCUUUGUUGGCCUACGATT | 68 |
| UCUUGGGCAACAGAUAACCAG | 29 | GGUUAUCUGUUGCCCAAGATT | 69 |
| UGAUAGAUCUAACAAAGGCAT | 30 | GCCUUUGUUAGAUCUAUCATT | 70 |
| UCUUAGGGAGGCUUAAAUCTG | 31 | GAUUUAAGCCUCCCUAAGATT | 71 |
| UUGGAAUAGGGAAACCCGGCA | 32 | CCGGGUUUCCCUAUUCCAATT | 72 |
| UAGUUGUCCAGCGCUCCCUCT | 33 | AGGGAGCGCUGGACAACUATT | 73 |
| UUCUCAUGUGGCACCUGACTG | 34 | GUCAGGUGCCACAUGAGAATT | 74 |
| UGGUUGGAGGACAUUCCUGAG | 35 | CAGGAAUGUCCUCCAACCATT | 75 |
| UUAUCUACUCAAAGCAUUAAA | 36 | UAAUGCUUUGAGUAGAUAATT | 76 |
| UUCUGGCACAACAGCAUCUCG | 37 | AGAUGCUGUUGUGCCAGAATT | 77 |
| UUCCACCAGGAGAGGCCCGGG | 38 | CGGGCCUCUCCUGGUGGAATT | 78 |
| UCUAAUCGUGCUCUUAUUCAA | 39 | GAAUAAGAGCACGAUUAGATT | 79 |
| UUACUUUAUUUGCAUCUCAGC | 40 | UGAGAUGCAAAUAAAGUAATT | 80 |
| UGAACGCCCGCCUCGAUCGGA | 41 | CGAUCGAGGCGGGCGUUCATT | 81 |
| UACUUUCCCAGGAUCCAGAGG | 42 | UGUGGAUCCUGGGAAAGUATT | 82 |
| AUACAAGCUCGUUUACAUGTG | 43 | CAUGUAAACGAGCUUGUAUTT | 83 |
| UACAAGCUCGUUUACAUGUGA | 44 | ACAUGUAAACGAGCUUGUATT | 84 |
| UACAUGUGAUAGAUCUAACAA | 45 | GUUAGAUCUAUCACAUGUATT | 85 |
| UGUGCAACAGUACUUUCCCAG | 46 | GGGAAAGUACUGUUGCACATT | 86 |
| UCGAGGUCAACAUUCUAGUTG | 47 | ACUAGAAUGUUGACCUCGATT | 87 |
| UCUAGUUGUCCAGCGCUCCCT | 48 | GGAGCGCUGGACAACUAGATT | 88 |
| AUUUGUAGAUCUCAGUGCCTA | 49 | GGCACUGAGAUCUACAAAUTT | 89 |
| UUUGCAGCCUUUGUGCAACAG | 50 | GUUGCACAAAGGCUGCAAATT | 90 |
| UCAAACAGGAUUGGAAUAGGG | 51 | CUAUUCCAAUCCUGUUUGATT | 91 |
| UGCAACAGUACUUUCCCAGGA | 52 | CUGGGAAAGUACUGUUGCATT | 92 |
| CUUAUAUUCAGUUUCCAAGTG | 53 | CUUGGAAACUGAAUAUAAGTT | 93 |
| AAAGGCACGAACACGUUCCAC | 54 | GGAACGUGUUCGUGCCUUUTT | 94 |
| UUUGUAGAUCUCAGUGCCUAT | 55 | AGGCACUGAGAUCUACAAATT | 95 |
| AAAGGCAUCUACCGAAGUCTG | 56 | GACUUCGGUAGAUGCCUUUTT | 96 |
| UUCUUGGGCAACAGAUAACCA | 57 | GUUAUCUGUUGCCCAAGAATT | 97 |
| UGCUGGUUGGAGGACAUUCCT | 58 | GAAUGUCCUCCAACCAGCATT | 98 |
| UUUCAAACAGGAUUGGAAUAG | 59 | AUUCCAAUCCUGUUUGAAATT | 99 |
| UUGCAUCUCAGCAAAGGUUCT | 60 | AACCUUUGCUGAGAUGCAATT | 100 |
| AAUCGUGCUCUUAUUCAACAT | S61 | GUUGAAUAAGAGCACGAUUTT | 101 |

The invention further relates to the use of an RNAi agent, such as an siRNA specific for cMAC, in the treatment of a disorder in a subject.

Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

### Peptide Mimetics

Peptide mimetics of cMAC proteins would also be predicted to act as cMAC modulators. Thus, one embodiment of this invention are peptides derived or designed from cMAC which block cMAC function. Suitable peptide mimetics to cMAC proteins can be made according to conventional methods based on an understanding of the regions in the polypeptides required for cMAC protein activity. Briefly, a short amino acid sequence is identified in a protein by conventional structure function studies such as deletion or mutation analysis of the wild-type protein. Once critical regions are identified, it is anticipated that if they correspond to a highly conserved portion of the protein that this region will be responsible for a critical function (such as protein-protein interaction). A small synthetic mimetic that is designed to look like said critical region would be predicted to compete with the intact protein and thus interfere with its function. The synthetic amino acid sequence could be composed of amino acids matching this region in whole or in part. Such amino acids could be replaced with other chemical structures resembling the original amino acids but imparting pharmacologically better properties, such as higher inhibitory activity, stability, half-life or bioavailability.

### Small Molecules

It is contemplated that modulators identified and discovered through the cMAC screening assays disclosed herein could be agents such as small molecules, including organic small molecules (with or without drug-like features), and including natural products. These small molecule modulators of cMAC include agonists of cMAC biological activity or cMAC expression; they may also include inhibitors of cMAC biological activity or inhibitors of cMAC expression. Those skilled in the art are familiar with screening of libraries of natural compounds, semi-synthetic compounds or combinatorial compound libraries, in low-throughput, medium-throughput, high-throughput and ultra-high-throughput formats. Compounds which are found to modulate cMAC activity, compared to control compounds, are identified, and grouped by chemical structure. The chemical structures are then modified and tested for further activity in the assay. The structure-activity relationship (SAR) of the compound to the target is evaluated. Compounds with high potency and high selectivity for the target are developed. Such compounds are then rigorously tested in a battery of other assays before being tested in animals and humans for therapeutic effect. All these steps are well known to those skilled in the art.

Further Research Uses of cMAC

It is also noted that the instant invention is useful for further research. For example, the cDNA encoding cMAC proteins and/or the cMAC proteins themselves can be used to identify other proteins, e.g. kinases, proteases or transcription factors, that are modified or indirectly activated in a cascade by cMAC proteins. Proteins thus identified can be used, for example, for drug screening to treat the pathological conditions discussed herein. To identify these genes that are downstream of cMAC proteins, it is contemplated, for example, that one could use conventional methods to treat animals in disease state models with a specific cMAC inhibitor, sacrifice the animals, remove relevant tissues and isolate total RNA from these cells and employ standard microarray assay technologies to identify message levels that are altered relative to a control animal (animal to whom no drug has been administered).

In addition, conventional in vitro or in vivo assays may be used to identify possible genes that lead to over expression of cMAC proteins. These related regulatory proteins encoded by genes thus identified can be used to screen drugs that might be potent therapeutics for the treatment of the pathological conditions discussed herein. For example, a conventional reporter gene assay could be used in which the promoter region of a cMAC protein is placed upstream of a reporter gene, the construct transfected into a suitable cell (for example from ATCC, Manassas, Va.) and using conventional techniques, the cells assayed for an upstream gene that causes activation of the cMAC promoter by detection of the expression of the reporter gene.

It is contemplated herein that one can inhibit the function and/or expression of a gene for a related regulatory protein or protein that modifies cMAC as a way to treat the pathological conditions discussed herein by designing, for example, antibodies to these proteins or peptide mimetics and/or designing inhibitory antisense oligonucleotides, triple helix DNA, ribozymes, siRNA, double or single stranded RNA and RNA aptamers targeted to the genes for such proteins according to conventional methods. Pharmaceutical compositions comprising such inhibitory substances for the treatment of said pathological conditions are also contemplated.

### Pharmaceutical Compositions and Administration

An additional embodiment of the invention relates to the administration of a pharmaceutical composition, in conjunction with a pharmaceutically acceptable carrier, excipient or diluent, for treatment of any of the pathological conditions discussed herein. Such pharmaceutical compositions may comprise any of the cMAC modulators disclosed herein, including the cMAC protein, or fragments thereof, antibodies to cMAC polypeptides, nucleic acids (e.g. gene therapy vectors, antisense, ribozyme or RNAi agents), cMAC peptide mimetics, small molecule modulators and any other cMAC modulators (e.g. agonists, antagonists, or inhibitors of cMAC expression and/or function). The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The instant invention further comprises a method of treating a disorder in a subject comprising administering to the subject an effective amount of an agent, or a pharmaceutical composition of an agent, that inhibits or enhances the activity or expression of cMAC.

Pharmaceutical compositions comprising cMAC modulators thereof may be administered when deemed medically beneficial by one of skill in the art, e.g. in conditions wherein agonists of cMAC function have a therapeutic effect such as neurodegenerative disorders such as Alzheimer's, Parkinson's and Huntington diseases. Such pharmaceutical compositions for use in accordance with the present invention may be formulated in a conventional manner using one or more physiologically acceptable carriers or excipients.

The pharmaceutical compositions disclosed herein as useful for preventing, treating or ameliorating pathological conditions disclosed herein are to be administered to a patient at therapeutically effective doses. A therapeutically effective dose refers to that amount of the compound sufficient to result in the prevention, treatment or amelioration of said conditions.

Compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient useful to prevent, treat or ameliorate a particular pathological condition of interest. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

It is contemplated herein that monitoring cMAC levels or activity and/or detecting cMAC expression (mRNA levels) may be used as part of a clinical testing procedure, for example, to determine the efficacy of a given treatment regimen. For example, patients to whom drugs have been administered would be evaluated and the clinician would be able to identify those patients in whom cMAC levels, activity and/or expression levels are higher than desired (i.e. levels higher or lower than levels in control patients not experiencing a related disease state or in patients in whom a disease state has been sufficiently alleviated by clinical intervention). Based on these data, the clinician could then adjust the dosage, administration regimen or type of medicinal prescribed.

Factors for consideration for optimizing a therapy for a patient include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary for the treatment of a given pathological condition.

The following examples further illustrate the present invention and are not intended to limit the invention.

### EXAMPLES

### General Methods

### TORC-1 Translocation Screen

The screen was performed as described (Bittinger et. al. Curr Biol. 2004 Dec. 14; 14(23):2156-61.) Briefly, a fluorescent fusion construct was created (Torc1-eGFP) and the construct was co-transfected with 7680 individual cDNA clones (predominantly from the MGC clone collection) into HeLa cells. The nuclear translocation of the Torc fusion protein was assessed using an automated fluorescence microscopy platform.

TORC-eGFP translocation quantitation: A stable expressing HeLa cell line expressing Torc1-GFP was prepared and cells were seeded (6000 cells per well in a 96 well plate), and transduced with lentiviral constructs (pLLB1)-GW-Kan) containing empty vector (translation stop sequence), TRPV6 or human cMAC. 48 hours post transduction the cells were treated with or without cyclosporine (5 μM) for one hour prior to fixing, imaging and quantitation using a Cellomics array scan II (fixing procedure see below). The difference between the nuclear cytoplasmic fluorescence intensity and the cytoplasmic fluorescence intensity was determined from 500 cell images per well.

Packaging of Moloney retrovirus expression particles: 24 hrs prior to transfection, 1.47×106 GP2-293 packaging cells were seeded on PDL plates (poly-d-lysine 6-well plates, Becton Dickinson) in 10% serum without antibiotics. 2.5 μg of expression construct QL-GW-final-kan vector DNA and 2.5 μg pvpackVSV-G plasmid (Stratagene) were combined in a final volume of 250 μl Optimem (Life Technologies). 12 μl Lipofectamine 2000 reagent was mixed in a final volume of 250 μl Optimem and incubated for 5 min at room temp. The diluted DNA was combined with the diluted lipofectamine (20 min at room temp). The complex was added to the GP2 cells (in 2 ml media without antibiotics) and incubated overnight. The following day the media was removed and replenished with fresh media containing antibiotics. 48 hrs post transfection the media containing the virus was collected and stored at 4° C.; cells were replenished with fresh media. 72 hrs post transfection, the final collection of virus was made and pooled with the previous (48 hr) collection sample. The virus supernatant was filtered through a 0.45 μM PVDF filter to remove any non-adherent cells and cellular debris.

Packaging of Lentiviral expression particles: 24 hrs prior to transfection of packaging constructs, 1.47×106 293T packaging cells were seeded on PDL plates (poly-d-lysine 6-well plates, Becton Dickinson) in 10% serum without antibiotics. 2 μg of lentiviral expression construct (pLLB1-GW-Kan) and 1 μg pLP-VSVG plasmid, 1 μg pLP1, 1 μg pLP2 (Invitrogen) suspended in 250 μl Optimem (Life Technologies). 12 μl Lipofectamine 2000 reagent was mixed in a final volume of 250 μl Optimem and incubated for 5 min at room temp. The diluted DNA was combined with the diluted lipofectamine (20 min at room temp). The complex was added to the 293 T cells (in 2 ml media without antibiotics) and incubated overnight. The following day the media was removed and replenished with fresh media containing antibiotics. 48 hrs post transfection the media containing the virus was collected and stored at 4° C.; cells were replenished with fresh media. 72 hrs post transfection, the final collection of virus was made and pooled with the previous (48 hr) collection sample. The virus supernatant was filtered through a 0.45 μM PVDF filter to remove any non-adherent cells and cellular debris.

Packaging of lentiviral shDNA constructs: The same general procedure described for the lentiviral expression constructs described with the following exceptions: 2.6×104 293T cells were seeded in 96 well plates 24 hrs prior to transfection. 100 ng shDNA construct (pLKO.1) and 10 ng pLP-vsvg plasmid, 50 ng pLP1, 50 ng pLP2 (Invitrogen) suspended in 30 μl Optimem (Life Technologies) and combined with 0.6 μL fugene6 (Roche). Complex was allowed to form for 30 minutes prior to addition to packaging cells.

### Description of Viral Constructs:

The pLL-B1-GW-Kan vector was derived from pLL3.7 vector from MIT lab (Luk VanParijs lab). A Gateway cassette was substituted for the eGFP marker and the U6 (shRNA promoter) was deleted. The kanamycin resistance cassette was substituted for the ampicillin cassette.

The QL-GW-final-Kan vector was derived from the pQCXIX vector from BD Biosciences. The CMV promoter and the IRES sequence were removed and a gateway cassette was inserted in the vector. The 3′LTR was substituted with a wild type 3′LTR which drives expression of the gene inserted.

The pLKO.1 shDNA lentiviral vector was unmodified and was obtained from The Broad Institute (The RNAi Consortium) in Cambridge Mass.

### NFAT Transcription Activation

HEK293 cells were transfected with 20 ng indicated plasmids in combination with 10 ng, Renilla, 20 ng pCMV-SPORT6, and 50 ng NFAT-Luc (Stratagene Inc). Transfections were carried out in 96 well format using approximately 20,000 cells per well. Cells were exposed to either DMSO, 5 μM CsA, 10 μM PMA, or 10 μM PMA and 5 μM CsA for 16 hours. Reporter activities were determined 72 hours after transfection with the Dual-Glo luciferase reagent as per manufacturer's instructions (Promega).

Fixing HeLa cells: Cells were fixed with 3.7% formaldehyde, 0.5% Triton X100 in PBS, 20 min at room temperature. Cells were washed twice with 0.5% Triton X100 in PBS.

Fixing and staining Jurkat cells, NFAT translocation assay: Following treatment Jurkat cells were attached to 96 well plates using the Becton Dickinson Cell-Tak cell adhesive. The suspension cells were centrifuged (800×G) for 5 min onto precoated plates (as per manufacturer's instruction). The attached cells were permeabilized in 3.7% formaldehyde, 0.5% Triton X100 and washed twice with wash buffer (0.5% Triton X100 in PBS) and blocked with 2% BSA, 0.1% Triton X100 in PBS. The cells were incubated in primary antibodies NFAT-1 (Cellomics K01-0011-1 diluted 1:100) and NFAT-2 (Affinity Bioreagents MA3-024 diluted 1:250) in blocking buffer for 1 hour at room temperature. Cells were washed twice with wash buffer and incubated with secondary goat anti-mouse IgG conjugated to Alexa Fluor 488 GαM (Cellomics K01-0011-1 reagents antibody diluted 1:2000, and Hoechst dye 1:2000) in blocking buffer. Cells were washed once with PBS and imaged.

Gateway transfer cDNA sequences into viral vectors: cDNA sequences of genes used in this study were obtained from the Gateway transfer of clones obtained from the MGC cDNA clone collection which were either used directly or transferred into viral vectors QL-GW-Kan/pLLB1-GW-Kan. This was accomplished using a single tube reaction and a two step reaction process. The BP reaction was performed by combining 100 ng pCMV-Sport6 cDNA plasmid with 100 ng pDONR207 (Invitrogen) intermediate plasmid. The reaction was initiated by adding 1.5 μL of BP 5× Clonase buffer (Invitrogen) and 1.5 μL BP Clonase (Invitrogen) in a total volume of 8 μL, room temperature overnight. LR reaction was performed by combining 4 μL BP reaction with 100 ng destination vector (QL-GW-Kan or pLLB1-GW-Kan) with 0.4 μL 0.75M NaCL, 1 μL 5×LR (Invitrogen) buffer and 1.8 μL LR Clonase in a final volume of 12 μL. Incubated at room temp overnight and transformed in STB3 cells (2 μL into 20 μL competent cells).

Transduction of HeLa cells. Cells were seeded 24 hrs prior to transduction, in clear bottom tissue culture-treated 96-well plates at 6000 cells/well (100 μl per well) in DMEM/FBS (10% Heat inactivated serum, Invitrogen) and antibiotic/antimycotic (1%, Invitrogen). Media was replaced with transduction media at a final concentration of 8 μg/ml polybrene (Sigma) and 10 mM HEPES buffer (Invitrogen). 50 μl of retroviral supernatant was added to each well and plates were centrifuged at 800×g for 90 min.

Transduction and sensitization of Jurkat cells: Jurkat cells were maintained in RPMI 1640 (GIBCO 21870-076) 10% FC1 Fetal clone1 (Hyclone), 1% Pen/Strep, 1% Glutamax1, 0.1% beta mercaptoethanol. Prior to transduction the cells were switched to transduction media which contains: RPMI 1640 (above) fortified with 2.25 g glucose 1% antibiotic/antimycotic, 1% 1M Hepes (Gibco), 1% 100 mM sodium pyruvate, 10 ml 7.5% Sodium Bicarbonate 10% Fetal Clone serum 1. Cells were seeded 5×104 in transduction media combined with virus (volume in legend) and 4 ug/ml polybrene final concentration and centrifuged at ˜800×g for 3 hrs. For activation of ICOS and IL-2 expression experiments cells were transduced with 50 μl of retroviral supernatant (QL-GW-Final-Kan) and 48 hours post transduction the media was fortified with PMA (phorbol 12-myristate 13-acetate) 10 ng/ml and 24 hours after addition the cells or media were removed for ICOS or IL-2 measurements. For NFAT translocation experiments cells were transduced with 50 μl of retroviral supernatant (pLL-B1-GW-Kan) and 48 hrs post transduction cells were sensitized with PMA 10 ng/ml for 6 hours prior to fixing and staining.

Analysis of ICOS surface marker and IL-2 protein expression: 1.5 uL of ICOS-PE (BD-Parmingen 557802) was combined with ˜1×10⁶ Jurkat cells and incubated on ice for 30 min. Cells were centrifuged ˜500×g 5 min and washed 2×PBS and the mean channel flourescence determined using flow cytometry (BD FACSCaliber). IL-2 levels were measured using the QuantiGlo IL-2 Elisa kit (R&D Systems).

Jurkat cell activation for shDNA inhibition studies: To assess shDNA efficacy, Jurkat cells (15000) were transduced with 10 μL LKO virus as described and 24 hours post transduction the media was fortified with puromycin (see below). Six days post transduction half the cells were activated with antibodies targeting TCR and CD28 receptors bound to a 96 well plate surface and incubated overnight and the media was collected for IL-2 determination. The remaining cells were used to determine the fraction of viable cells in each well using the Cell Titer-Glo assay (see below).

The activation plates were prepared by coating goat anti-mouse IgG, Fcγ fragment specific antibody (Jackson ImmunoResearch Laboratories) 55 μl per well at a final concentration of 10 μg/ml in PBS. The plates were incubated 3 hours at room temperature. Excess IgG was removed and plates were blotted. Plates were blocked with 300 μL 2% BSA/PBS (BSA, Fraction V lyophilizate, Roche) and incubated for 2 hours at room temperature. The plates were washed 3 times with PBS and stimulating antibodies anti-TCR 0.01 ug/ml (BD Biosciences 347770 clone WT31) and anti-CD28 0.3 ug/ml (BD Pharmingen 555725) in 2% BSA/PBS final volume 50 μL/well. Plates were incubated overnight and washed 3 times with PBS prior to addition of cells for activation.

Puromycin selection of shDNA transduced Jurkat cells and normalization for cell survival: The LKO viral vector used in this study contains a puromycin selection marker. Jurkat cells infected with shDNA constructs (LKO.1) were put under puromycin selection 24 hrs post infection by addition of puromycin (2 μg/ml) and maintained for the duration of the experiment (6 days post infection, 3 days for mRNA quantitation). To account for differences in cell number after puromycin selection (possibly due to variations in viral titer), we adopted a cellular ATP assay which is proportional to cell number (Cell Titer-Glo Luminescent Assay Kit, Promega). The assay was performed as per the manufacturer's instruction. A standard curve was generated for each cell type to determine linearity for the assay. The IL-2 concentrations were normalized to cell number by dividing the calculated IL-2 concentration by the rLU values for the cell titer-glo assay, which is equivalent to IL-2 expression/cell number. The mRNA expression levels were determined 3 days post infection.

Determination of mRNA knockdown for cMAC: Jurkat mRNA expression levels were determined 3 days post infection (2 days post puromycin selection).

Preparation of shDNA constructs and ligation into LKO.1 vector: DNA oligos were synthesized with adapters for 5′ Agel and 3′ EcoR1 with loop sequence TTCAAGAGA. Oligos were annealed and ligated directly into predigested LKO vector. See Table 6 for Oligo sequences.

TABLE 6
shDNA target sequence and oligos ligated into LKO.1 vector

| | | |
|---|---|---|
| Target sequence | Sense DNA oligo | Antisense DNA oligo |

| | | | |
|---|---|---|---|
| cMAC BL1 | gcgcctgtcgggtcaacta | ccgggcgcctgtcgggtcaactattca | aattaaaaagcgcctgtcgggtcaact |
| | (SEQ ID NO: 102) | agagatagttgacccgacaggcgcttt | atctcttgaatagttgacccgacaggc |
| | | tt (SEQ ID NO: 103) | gc (SEQ ID NO: 104) |
| cMAC BL2 | gcctttgttagatctatca | ccgggcctttgttagatctatcattcaag | aattaaaaagcctttgttagatctatcat |
| | (SEQ ID NO: 105) | agagatgatagatctaacaaaggcttttt | ctcttgaatgatagatctaacaaaggc |
| | | (SEQ ID NO: 106) | (SEQ ID NO: 107) |
| cMAC BL3 | catctttgttggcctacga | ccggcatctttgttggcctacgattcaa | aattaaaaacatctttgttggcctacgat |
| | (SEQ ID NO: 108) | gagatcgtaggccaacaaagatgttttt | ctcttgaatcgtaggccaacaaagatg |
| | | (SEQ ID NO: 109) | (SEQ ID NO: 110) |
| cMAC BL4 | ggttatctgttgcccaaga | ccggggttatctgttgcccaagattcaa | aattaaaaaggttatctgttgcccaaga |
| | (SEQ ID NO: 111) | gagatcttgggcaacagataaccttttt | tctcttgaatcttgggcaacagataacc |
| | | (SEQ ID NO: 112) | (SEQ ID NO: 113) |
| cMAC BL5 | gatttaagcctccctaaga | ccgggatttaagcctccctaagattcaa | aattaaaaagatttaagcctccctaaga |
| | (SEQ ID NO: 114) | gagatcttagggaggcttaaatcttttt | tctcttgaatcttagggaggcttaaatc |
| | | (SEQ ID NO: 115) | (SEQ ID NO: 116) |
| cMAC BL6 | actagaatgttgacctcga | ccggactagaatgttgacctcgattca | aattaaaaaactagaatgttgacctcga |
| | (SEQ ID NO: 117) | agagatcgaggtcaacattctagtttttt | tctcttgaatcgaggtcaacattctagt |
| | | (SEQ ID NO: 118) | (SEQ ID NO: 119) |
| cMAC BL7 | ccgggtttccctattccaa | ccggccgggtttccctattccaattcaa | aattaaaaaccgggtttccctattccaat |
| | (SEQ ID NO: 120) | gagattggaatagggaaacccggtttt | ctcttgaattggaatagggaaacccgg |
| | | t (SEQ ID NO: 121) | (SEQ ID NO: 122) |
| cMAC BL8 | gtcaggtgccacatgagaa | ccgggtcaggtgccacatgagaattc | aattaaaaagtcaggtgccacatgaga |
| | (SEQ ID NO: 123) | aagagattctcatgtggcacctgactttt | atctcttgaattctcatgtggcacctgac |
| | | t (SEQ ID NO: 124) | (SEQ ID NO: 125) |
| cMAC BL9 | caggaatgtcctccaacca | ccggcaggaatgtcctccaaccattca | aattaaaaacaggaatgtcctccaacc |
| | (SEQ ID NO: 126) | agagatggttggaggacattcctgttttt | atctcttgaatggttggaggacattcct |
| | | (SEQ ID NO: 127) | g (SEQ ID NO: 128) |
| cMAC MB4 | ccttgggcuggcactcttatt | ccggccttgggcuggcactcttattca | aattaaaaaccttgggctggcactctta |
| | (SEQ ID NO: 129) | agagataagagtgccagcccaaggtt | tctcttgaataagagtgccagcccaag |
| | | ttt (SEQ ID NO: 130) | g (SEQ ID NO: 131) |
| CD29 | ggtagaaagtcgggacaaa | ccggggtagaaagtcgggacaaattc | aattaaaaaggtagaaagtcgggaca |
| | (SEQ ID NO: 132) | aagagatttgtcccgactttctaccttttt | aatctcttgaatttgtcccgactttctacc |
| | | (SEQ ID NO: 133) | (SEQ ID NO: 134) |
| pGL3-Luc | cttacgctgagtacttcga | ccggcttacgctgagtacttcgattcaa | aattaaaaacttacgctgagtacttcga |
| | (SEQ ID NO: 135) | gagatcgaagtactcagcgtaagttttt | tctcttgaatcgaagtactcagcgtaag |
| | | (SEQ ID NO: 136) | (SEQ ID NO: 137) |

### Example 1

### Discovery that cMAC Induces Nuclear Translocation of TORC1

NFAT, NFkB and AP-1 are probably the three most important transcription factors in the activation of T-cells (Quintana Eur J Physiol (2005) 450:1-12). All three promoter elements are represented on the IL-2 promoter and all three have been determined to be calcium dependent (NFkB and AP-1 indirectly). The activation of T-cells requires NFAT translocation into the nucleus. This is mediated by the unmasking of the nuclear localization sequence on NFAT by the phosphatase enzyme calcineurin. Calcineurin is activated through calcium mobilization and is blocked by the immunosuppressive drug cyclosporine A. TORC-1 is a cAMP dependent coactivator of transcription. TORC-1 is similar to NFAT in that Torc-1 is translocated into the nucleus upon activation following calcium mobilization. TRPV6 is thought to be a store-operated calcium channel and has, although controversial, been suggested to have similarity to the calcium release-activated calcium (CRAC) channel which is responsible for induction of calcium activation-regulated genes (Feske Nat. Immunol. 2(4):316-24 (2001)).

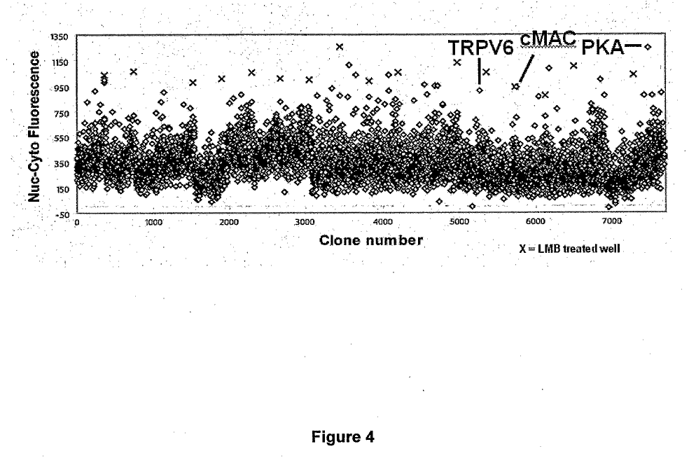

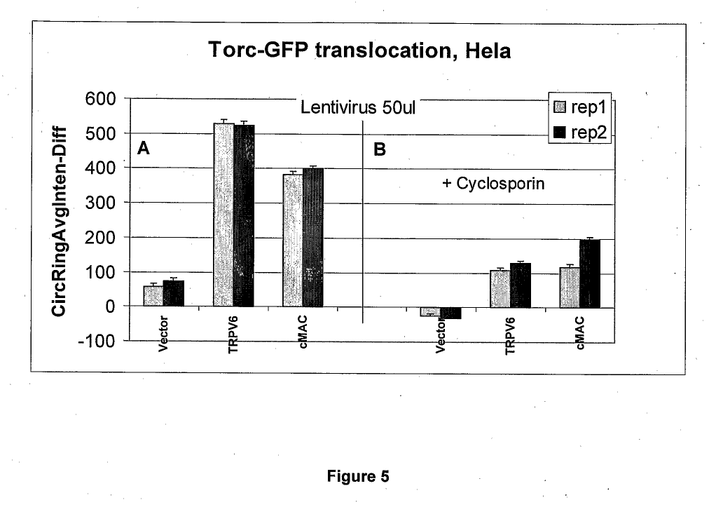

A high content imaging screen to identify genes which were involved in the translocation of the cAMP dependent CREB co-activator TORC1 has been performed. This screen identified a number of genes as inducers of TORC nuclear translocation FIG. 4 and Table 7 (Bittinger et. al. Current Biology 14(23):2156-61 (2004)), however the identity of one previously uncharacterized gene which we now refer to as cMAC (conserved Membrane Activator of Calcineurin) was not disclosed in that publication. The published sequence of murine cMAC (Accession NM_{—}177344) and the human ortholog cMAC (Accession NM_{—}053045) are found in GenBank. The cMAC clone found in the screen was an MGC clone which was annotated as being similar to NM_{—}177344. However, NM_{—}177344 encodes a protein with an alternative 3′ end which is not present in human cDNAs or in the predicted orthologs of cMAC. The cDNAs active in the primary screen as well as the human ortholog of murine cMAC were retrieved, retransformed, sequence confirmed, and inserted into viral vectors and introduced into HeLa cells stably expressing TORC1-eGFP, and the relative amounts of TORC1-eGFP in the cytosol and nucleus were calculated using an automated microscopy platform in example 2 below. The Torc-eGFP translocation was blocked by the calcineurin inhibitor cyclosporine A which also illustrates that the cDNA's indeed induced TORC translocation as opposed to affecting cell morphology or other phenotypes which may be misinterpreted by the automated microscopy platform as translocation.

TABLE 7
Putative inducers of TORC translocation

| MGC | | Abbrevi- |
|---|---|---|
| designation | Annotation | ation |
| BC022606, | Mus musculus RIKEN cDNA C730025P13 | cMAC |
| 15-P2 | gene, mRNA (cDNA clone MGC: 31129 | |
| | IMAGE: 4165766), complete cds. | |
| | NM_177344 | |
| BC034814 | Homo sapiens transient receptor | TRPV6 |
| | potential cation channel, subfamily | |
| | V, member 6 (TRPV6), mRNA. NM_018646 | |

### Example 2

### cMAC Acts Via Calcium and Calcineurin

To determine if TORC translocation by cMAC was through calcium and calcineurin, cMAC was over expressed in HeLa cells which stably express the Torc1-eGFP fusion construct by infecting the cells with lentiviral particles containing the calcium channel TRPV6 and the human cMAC sequence. Cells were treated with the calcineirin inhibitor Cyclosporin A (CsA) one hour prior to imaging. As shown in FIG. 5: CsA treatment resulted in a reversal of TORC1 translocation, resulting in most of the TORC1-eGFP being returned to the cytoplasm. These data suggest that cMAC translocation of TORC-1 was dependent on calcineurin activation. In a separate set of experiments, a requirement for extracellular calcium was also tested using EGTA. The presence of EGTA in the medium blocked cMAC induced nuclear TORC1 translocation. The effect of EGTA could be reversed however by addition of excess calcium to the medium (data not shown). Thus cMAC induces TORC1 translocation through a calcium-dependent activation of calcineurin.

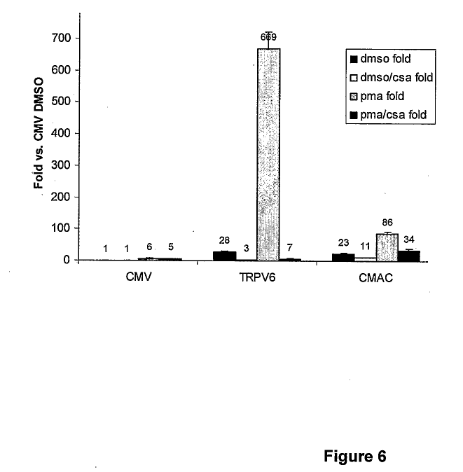

The effect of cMAC overexpression on the calcineruin dependent NFAT transcription factor was also examined. cMAC or the previously described NFAT activator TRPV6 were co-transfected with an NFAT driven luciferase reporter. In the presence of PMA, cMAC (and TRPV6) overexpression induced a significant increase in NFAT driven expression (FIG. 6) and this activation was partially blocked by CsA. This data is consistent with activation of calcineurin. It should be noted that activation by cMAC was not nearly as strong as that by the calcium channel TRPV6.

### Example 3

### Effect of cMAC on Nuclear Translocation of NFAT and Activation of T-Cells

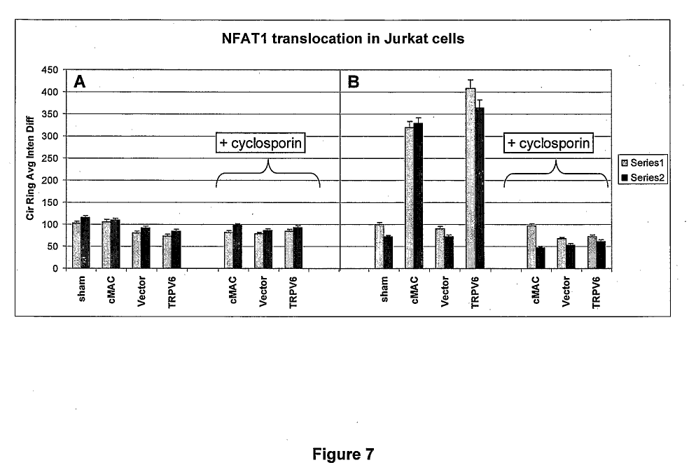

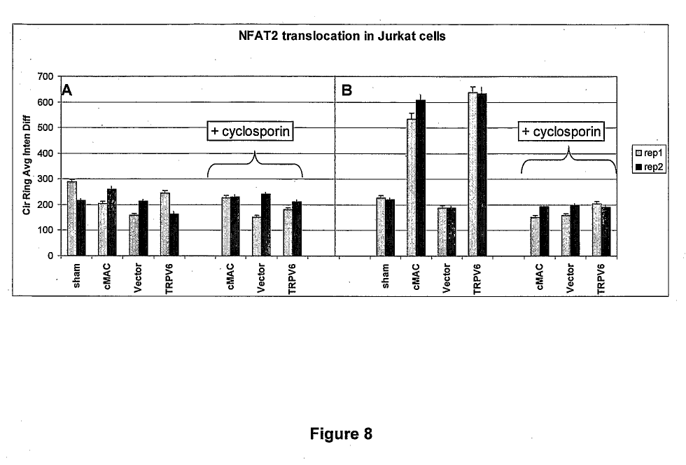

The effect of cMAC on the nuclear translocation of the transcription factor NFAT and its dependence on calcineurin was also examined. Jurkat cells were transduced with an empty viral vector (translation stop sequence), the calcium channel TRPV6 or human cMAC. Cells were examined for the location of endogenous NFAT-1 (FIG. 7) and NFAT-2 (FIG. 8) protein. While NFAT-1 and NFAT-2 were detected in the cytoplasm of control virus infected cells, both isoforms of NFAT were localized primarily in the nucleus in a large proportion of cMAC transduced cells. The translocation was dependent on sensitization of the cells with PMA. PMA alone demonstrated no effect on NFAT translocation however PMA in combination with cMAC or TRPV6 dramatically increased nuclear translocation. The calcineurin inhibitor cyclosporine A blocked the PMA sensitized cMAC (and TRPV6) induced translocation of both NFAT-1 and NFAT-2.

Calcium mobilization and subsequent NFAT translocation is a critical component in the signal transduction pathway in the activation of T-cells and NFAT is required for the production of IL-2. These data further support the role of NFAT and calcineurin in the activation of T-cells and specifically demonstrate that cMAC over expression in a T-cell line induces calcineurin dependent endogenous NFAT-1 and NFAT-2 translocation.

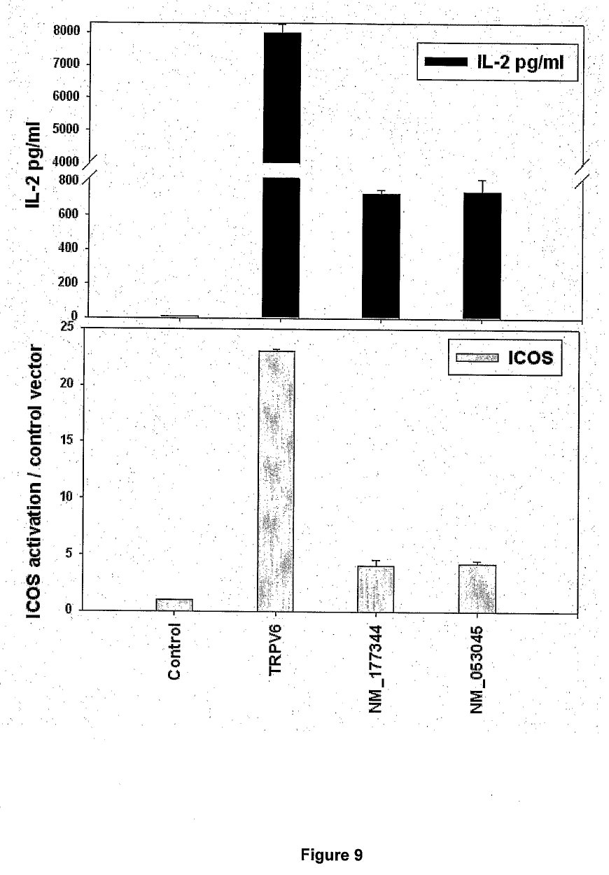

We assessed the role of TRPV6 and cMAC in a T-cell activation screen. Jurkat T-cells were transduced with TRPV6 and cMAC (FIG. 9) and tested for their ability to induce T-cell activation markers after priming with anti-TCR antibody and PMA (similar results were obtained with PMA alone). Both TRPV6 and cMAC were potent activators of T-cells as assessed by the induction of IL-2 and the expression of the surface marker ICOS, while PMA alone or PMA plus anti-TCR did not upregulate IL-2 or ICOS expression levels. Conversely, expression of the cDNAs demonstrated insignificant upregulation of IL-2 and ICOS without PMA sensitization, which is consistent with the findings observed with the translocation of NFAT. In this assay cDNAs encoding both human and mouse cMAC cDNAs, annotated as matching the RefSeq sequences NM_{—}0539045 and NM_{—}177344, respectively, were tested. Both species of cMAC cDNAs were similarly active in inducing IL-2 secretion and ICOS expression. Thus, cMAC overexpression also induces T-cell activation markers.

### Example 4

### Use of shRNA to Demonstrate cMAC is Critical to Activation of Jurkat T-Cells

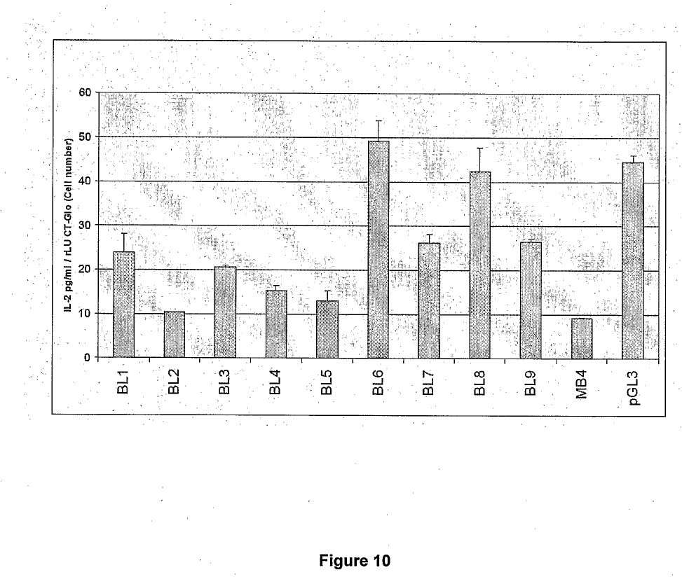

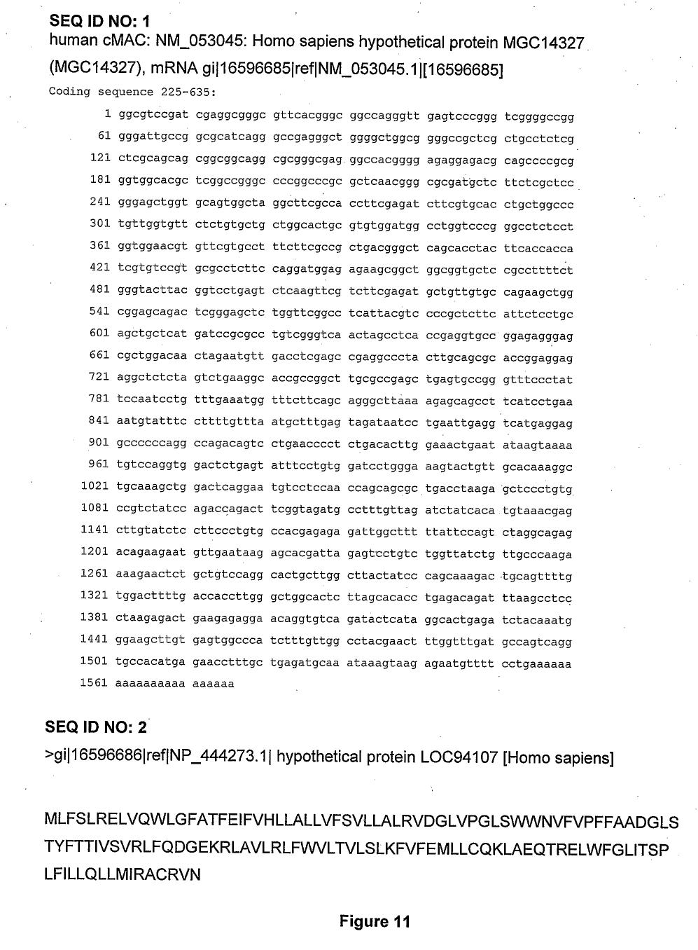

To assess whether cMAC was essential for T-cell activation, we designed several shDNA constructs targeting cMAC. In these experiments Jurkat T-cells were transduced with viral constructs targeting cMAC or another unrelated gene. 6 days post transduction, the cells were activated with TCR/CD28 antibodies and the level of cMAC mRNA and IL-2 secreted into the media was measured. All but two shDNA constructs targeting cMAC demonstrated significant reductions in IL-2 production. FIG. 10. In separate experiments, other negative controls targeting CD29 protein and a random murine gene demonstrated a similar inhibition profile as the negative control pGL3.

To determine the specificity of the shDNA construct, the reduction of cMAC mRNA was measured for each construct (Table 8) and compared to the inhibition of IL-2 observed. The pGL3-Luc and CD29 shDNA constructs served as negative controls. The CD29 shDNA construct potently reduced CD29 mRNA (and CD29 protein; data not shown) but had no effect on cMAC message. Only one construct (BL8) demonstrated satisfactory message knockdown (70%) without any IL-2 reduction, a second construct (BL6) demonstrated marginal mRNA knockdown (36%) without any IL-2 reduction. The remaining constructs demonstrated significant IL-2 decreases with mRNA reduction although in some instances mRNA reductions were marginal. In those instances it may be that the shDNA is causing decreases in the expression of cMAC protein through microRNA effects. Thus, it appears that with the exception of 2 constructs (BL6 and BL8) the phenotypically active IL-2 constructs correlate with reduced cMAC mRNA levels.

TABLE 8
Viral mediated shDNA knockdown of cMAC
mRNA correlates with IL-2 inhibition

| | Average IL-2 | | Average cMAC | |
|---|---|---|---|---|
| Construct | % inhibition | SEM | mRNA % Inhib. | SEM |
| cMAC BL1 | 44.4% | 9.77% | 20.4% | 4.80% |
| cMAC BL2 | 75.5% | 0.07% | 72.6% | 2.05% |
| cMAC BL3 | 52.1% | 0.25% | 30.1% | 7.15% |
| cMAC BL4 | 65.7% | 1.31% | 56.2% | 2.60% |
| cMAC BL5 | 72.2% | 2.49% | 67.0% | 1.40% |
| cMAC BL6 | −9.34% | 5.29% | 35.8% | 7.65% |
| cMAC BL7 | 40.8% | 2.04% | 66.4% | 1.75% |
| cMAC BL8 | 8.03% | 6.43% | 70.1% | 1.45% |
| cMAC BL9 | 37.8% | 0.72% | 54.8% | 6.20% |
| cMAC MB4 | 78.9% | 0.05% | 65.9% | 1.15% |

1. An isolated polypeptide of SEQ ID NO: 2, or a fragment thereof, or a substantially similar protein sequence having sequence identity of at least 50% with SEQ ID NO: 2, or a functional equivalent thereof, and exhibiting a biological activity selected from ion transport, ion diffusion, calcineurin pathway activation, calcium dependent activation of a T-cell, nuclear translocation of TORC, nuclear translocation of NFAT or cAMP Response Element (CRE)-driven gene expression activity of native SEQ ID NO: 2.

2. The polypeptide of claim 1 having a sequence selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

3. An antibody or antibody fragment that is capable of binding the polypeptide of claim 1.

4. An antibody or antibody fragment that binds specifically to cMAC (SEQ ID NO. 2), or a polypeptide comprising a cMAC-specific binding region.

5. An antibody fragment according to claim 3 which is an Fab or F(ab′)2 fragment.

6. An antibody according to claim 3 which is a monoclonal antibody.

7. An isolated nucleic acid molecule encoding the polypeptide of claim 1.

8. The nucleic acid molecule of claim 7 comprising the SEQ ID NO: 1, SEQ ID NO: 11 or SEQ ID NO: 12.

9. The nucleic acid molecule of claim 7 further comprising a promoter operably linked to the nucleic acid molecule.

10. An isolated nucleic acid sequence selected from among SEQ ID NOs 3, 4 and 5.

11. A vector molecule comprising the nucleic acid molecule of claim 7.

12. A vector molecule of claim 11 comprising the nucleic acid sequence of cMAC (SEQ ID NO. 1).

13. A vector comprising the promoter of cMAC (SEQ ID NO: 3) operably linked to a reporter protein nucleic acid sequence.

14. A host cell comprising the vector molecule of claim 11.

15. A method for producing the polypeptide of claim 1 comprising culturing the host cell having incorporated therein an expression vector comprising the vector of claim 11 under conditions sufficient for expression of the polypeptide in the host cell.

16. A method for producing a cMAC polypeptide of SEQ ID NO. 2 comprising culturing the host cell having incorporated therein an expression vector comprising the vector of claim 11 under conditions sufficient for expression of the polypeptide in the host cell.

17. A method of treating a disorder in a subject comprising administering to the subject an effective amount of an agent that inhibits the activity of cMAC.

18. A method according to claim 17 wherein the disorder is a cMAC-associated disorder.

19. A method according to claim 17 wherein said agent is antibody, an antibody fragment or a polypeptide containing a cMAC-specific binding region.

20. An antibody, an antibody fragment or a polypeptide of claim 3 comprising a cMAC-specific binding region as a medicament.

21-23. (canceled)

24. A method of treating a disorder in a subject comprising administering to the subject an effective amount of an agent that inhibits the expression of cMAC.

25. A method according to claim 24 wherein the disorder is a cMAC-associated disorder.

26. A method according to claim 24 wherein said agent is an inhibitory nucleic acid capable of specifically inhibiting expression of cMAC.

27. A method according to claim 26 wherein said inhibitory nucleic acid is selected from among the group consisting of an antisense oligonucleotide, an RNAi agent, and a ribozyme.

28. A method according to claim 27, wherein the RNAi agent is selected from among the group consisting of dsRNA, siRNA, and shRNA.

29. The method according to claim 28, wherein the RNAi agent comprises at least one nucleic acid selected from the group consisting of SEQ ID NO: 22 to SEQ ID NO: 101, and SEQ ID NO: 106, SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131, SEQ ID NO: 133, SEQ ID NO: 134, SEQ ID NO: 136, and SEQ ID NO: 137.

30. An RNAi agent comprising at least one nucleic acid selected from the group consisting of SEQ ID NO: 22 to SEQ ID NO: 101, and SEQ ID NO: 106, SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131, SEQ ID NO: 133, SEQ ID NO: 134, SEQ ID NO: 136, and SEQ ID NO: 137.

31. An RNAi agent specific for cMAC selected from among the group consisting of dsRNA, siRNA, and shRNA as a medicament, wherein the RNAi agent comprises at least one nucleic acid selected from the group consisting of SEQ ID NO: 22 to SEQ ID NO: 101, and SEQ ID NO: 106, SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131, SEQ ID NO: 133, SEQ ID NO: 134, SEQ ID NO: 136, and SEQ ID NO: 137.

32-34. (canceled)

35. A method of treating a disorder in a subject comprising administering to the subject an effective amount of an agent that enhances the activity of cMAC.

36. A method of treating a disorder in a subject comprising administering to the subject an effective amount of an agent that increases the expression of cMAC.

37. A method according to claim 36 wherein said agent is an enhancer of cMAC gene transcription.

38. A method according to claim 36 wherein said agent is a gene therapy vector comprising a nucleic acid encoding CMAC or a fragment thereof.

39. The method of claim 38 wherein said agent is a vector of claim 11.

40. A method according to claim 36 wherein the disorder is a cMAC-associated disorder.

41. A pharmaceutical composition comprising an effective amount of an agent which inhibits the expression of cMAC or inhibits an activity of cMAC, and a pharmaceutically acceptable carrier.

42. A pharmaceutical composition according to claim 41 wherein the agent is an antisense oligonucleotide or an RNAi agent.

43. The pharmaceutical composition according to claim 42 wherein the RNAi agent comprises at least one nucleic acid selected from the group consisting of SEQ ID NO: 22 to SEQ ID NO: 101, and SEQ ID NO: 106, SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131, SEQ ID NO: 133, SEQ ID NO: 134, SEQ ID NO: 136, and SEQ ID NO: 137.

44. A pharmaceutical composition according to claim 41 wherein the agent is an antibody an antibody fragment which binds specifically to cMAC, or a polypeptide comprising a cMAC-specific binding region.

45. A pharmaceutical composition of claim 44 wherein the antibody is the antibody of claim 3.

46. A pharmaceutical composition according to claim 44 wherein the agent binds b an epitope of cMAC selected from among SEQ ID NO. 6, 7, 8, 9, 10.

47. A method of treating a disorder in a subject comprising administering to the subject an effective amount of a pharmaceutical composition of an agent that inhibits the activity of cMAC.

48. A method according to claim 47 wherein the disorder is a cMAC-associated disorder.

49. A method according to claim 47 wherein said agent is an antibody or fragment thereof which binds specifically to cMAC (SEQ ID NO:2) or a polypeptide comprising a cMAC-specific binding region.

50. A method of claim 49 wherein the antibody is the antibody of claim 3.

51. A method according to claim 49 wherein the agent binds to an epitope of cMAC selected from among SEQ ID NOs. 6, 7, 8, 9 and 10.

52. A method of treating a disorder in a subject comprising administering to the subject an effective amount of a pharmaceutical composition of an agent that inhibits the expression of cMAC.

53. A method according to claim 52 wherein said agent is an inhibitory nucleic acid capable of specifically inhibiting expression of cMAC.

54. A method according to claim 53 wherein said inhibitory nucleic acid is selected from among the group consisting of an antisense oligonucleotide, an RNAi agent, and a ribozyme.

55. A method according to claim 54 wherein the RNAi agent is selected from among the group consisting of dsRNA, siRNA, and shRNA.

56. A method according to claim 55 wherein the RNAi agent comprises at least one nucleic acid selected from the group consisting of SEQ ID NO: 22 to SEQ ID NO: 101, and SEQ ID NO: 106, SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118., SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131, SEQ ID NO: 133, SEQ ID NO: 134, SEQ ID NO: 136, and SEQ ID NO: 137.

57. A method for identifying a compound useful for the treatment of a cMAC-associated disorder comprising:
(a) contacting a test compound with cMAC; and
(b) detecting a change of a biological activity of cMAC compared to cMAC not contacted with the test compound,
wherein detecting a change identifies said test compound as useful for the treatment of said disorder.

58. A method of identifying a compound useful for treatment of a cMAC-associated disorder comprising:
(a) contacting a test compound with cMAC under sample conditions permissive for cMAC biological activity;
(b) determining the level of a cMAC biological activity;
(c) comparing said level to that of a control sample lacking said test compound; and,
(d) selecting a test compound which causes said level to change for further testing as a potential agent for treatment of said disorder.

59. A method according to claim 57 wherein the said change is a reduction of such biological activity.

60. A method according to claim 57 wherein said biological activity is selected from among ion transport, ion diffusion, protein-cMAC interaction or cMAC modification, calcium dependent activation of a T-cell, nuclear translocation of TORC, and CAMP Response Element (CRE)-driven gene expression.

61. A method for testing if a compound modulates a cMAC biological activity comprising:
(a) contacting a test compound with cMAC; and
(b) detecting a change of a biological activity of cMAC compared to cMAC not contacted with the test compound,
wherein detecting a change identifies said test compound as a modulator of cMAC biological activity.

62. A method to identify modulators useful to treat a disorder comprising assaying for the ability of a candidate modulator to inhibit the activity of a cMAC protein.

63. A method to identify modulators useful to treat a disorder comprising assaying for the ability of a candidate modulator to inhibit the expression of a cMAC protein.

64. A compound identified by a method according to claim 57.

65. A method for identifying a compound useful for the treatment of a cMAC-associated disorder comprising administering a compound identified by a method according to claim 65 to an animal model of said cMAC-associated disorder.

66. The method according to claim 18, wherein the cMAC-associated disorder is selected from among the group consisting of autoimmune disease, immunosuppression, inflammatory disease, cancer, cardiovascular disease and neurological disease.

67. A method of inhibiting cMAC biological activity in a cell comprising contacting a cell with an anti-cMAC antibody or fragment thereof, with a polypeptide comprising a cMAC-specific binding region or with a nucleic acid which reduces cMAC expression.

68. A method according to claim 67 wherein said biological activity is selected from among the group consisting of calcium dependent activation of a T-cell, nuclear translocation of TORC, nuclear translocation of NFAT and CAMP Response Element (CRE)-driven gene expression.

69. A method of selectively inhibiting lymphocyte activity in a multi-cellular organism comprising contacting said organism with an anti-cMAC antibody or fragment thereof, with a polypeptide comprising a cMAC-specific binding region or with a nucleic acid which reduces cMAC expression.

70. A method of enhancing T-cell activation comprising contacting a T-cell or a T-cell precursor cell with a purified cMAC polypeptide, a gene therapy vector comprising the cMAC gene, or an enhancer of cMAC gene expression.

71. The method of claim 70 wherein the cMAC polypeptide is the polypeptide of claim 1.

72. The method of claim 70 wherein gene therapy vector comprising the cMAC gene is the vector of claim 11.


SEQUENCE LISTING (137 sequences)

SEQ ID NO: 1 - DNA, 1576 bp, Homo sapiens
ggcgtccgatcgaggcgggcgttcacgggcggccagggttgagtcccgggtcggggccgggggattgccggcgcatcagggccgagggctggggctggcggggccgctcgctgcctctcgctcgcagcagcggcggcaggcgcgggcgagggccacggggagaggagacgcagccccgcgggtggcacgctcggccgggccccggcccgcgctcaacgggcgcgatgctcttctcgctccgggagctggtgcagtggctaggcttcgccaccttcgagatcttcgtgcacctgctggccctgttggtgttctctgtgctgctggcactgcgtgtggatggcctggtcccgggcctctcctggtggaacgtgttcgtgcctttcttcgccgctgacgggctcagcacctacttcaccaccatcgtgtccgtgcgcctcttccaggatggagagaagcggctggcggtgctccgccttttctgggtacttacggtcctgagtctcaagttcgtcttcgagatgctgttgtgccagaagctggcggagcagactcgggagctctggttcggcctcattacgtccccgctcttcattctcctgcagctgctcatgatccgcgcctgtcgggtcaactagcctcaccgaggtgccggagagggagcgctggacaactagaatgttgacctcgagccgaggccctacttgcagcgcaccggaggagaggctctctagtctgaaggcaccgccggcttgcgccgagctgagtgccgggtttccctattccaatcctgtttgaaatggtttcttcagcagggcttaaaagagcagccttcatcctgaaaatgtatttccttttgtttaatgctttgagtagataatcctgaattgaggtcatgaggaggccccccaggccagacagtcctgaacccctctgacacttggaaactgaatataagtaaaatgtccaggtggactctgagtatttcctgtggatcctgggaaagtactgttgcacaaaggctgcaaagctggactcaggaatgtcctccaaccagcagcgctgacctaagagctccctgtgccgtctatccagaccagacttcggtagatgcctttgttagatctatcacatgtaaacgagcttgtatctccttccctgtgccacgagagagattggctttttattccagtctaggcagagacagaagaatgttgaataagagcacgattagagtcctgtctggttatctgttgcccaagaaaagaactctgctgtccaggcactgcttggcttactatcccagcaaagactgcagttttgtggacttttgaccaccttgggctggcactcttagcacacctgagacagatttaagcctccctaagagactgaagagaggaacaggtgtcagatactcataggcactgagatctacaaatgggaagcttgtgagtggcccatctttgttggcctacgaactttggtttgatgccagtcaggtgccacatgagaacctttgctgagatgcaaataaagtaagagaatgttttcctgaaaaaaaaaaaaaaaaaaaaaa

SEQ ID NO: 2 - protein, 136 aa, Homo sapiens
MLFSLRELVQWLGFATFEIFVHLLALLVFSVLLALRVDGLVPGLSWWNVFVPFFAADGLSTYFTTIVSVRLFQDGEKRLAVLRLFWVLTVLSLKFVFEMLLCQKLAEQTRELWFGLITSPLFILLQLLMIRACRVN

SEQ ID NO: 3 - DNA, 3101 bp, Homo sapiens
tcttgaactcctgggctcaagcgatcctcccacctcggcctcccaaattgctgggattacaggcatgagccactgtgcccggcaaacgtctctcataaaaaactccttctttttaactctttagccttttcacagccagatgtggttttttgtttgtttgttttgttttttgtcttttttttgtttttgagacggagtctcgctctgtcgcccaggctggagtgcagtggcgcgatctcggctcactgcaagttccgcctcccgggttcacgccattctcctgcctcagcctcccgagtagctgggactacaggtgcccgccaccacatccggctagttttttgtatttttagtagagacgaggtttcacaccgtgttagccaggatggtctcgatctcctgacctcgtgatctgcctgcctcggcctcccaaagtgctaggattacaggtgtgagccacagtgcccggctctttttctttttttttttttttttgacagagtctagctctgtcaccagtctggagtgcagtggcgcaatctcagctcactgcaacttccgactccctggtctaagtgattctcctgcctcagcctcccgagtagctgggattacaggcacgcaccactgcgccgtgcccagctaattgttgtaattttagtagagacgggtttcaccatgttggccaggctggtctcgaactcctgacctcgtgattcgcccgcctcggcctcccaaagtgctgagattacaggcgtgagccaccgcagccggcccgcactcaagacattcttaaaagatgctctccactcactctctcagttgctgatctcctgtaatctggcttctctccccatgagccactgaagctgctcttcctggtatcaccaacaatccctttgccaagaaatcccatggatccctctcaggctttatctgatttgacttctgtgcagctttacatgccagagcccttcctggaacactcactcccccagtcctctccttggggtgctgcaccctctcttctagctgcttggtcactctcctgccctcctgggctctaacgttctgggcagcctgtcctgtggggagagaggagcttcctcttctcacacttctgtattgtggctacctcaacacctatctccgccccaggatcttccttggagcgctagtcctactgcacttctccacttcaatgtctccaggttcccttcctgcacagccccctactcccaccgtgttctctgatttctgtggatggaaccaccatccatcttggttccagagcccacatccctgtctcgattctgcttgacctattgggtccgttccatgtcccggggactctctcttcctgacaccactgcaatgtgtccacttctatcttctgccaccacttcagctcagatcggtagcatctgctgcctgtcctgtgcatctcatggcattattcctgctgtcctgcagaccagtgcggttttgctagcttaggaacatgactgtgtcacccctacagttccgacaacccttcatttcaagcccatctgccttgctgtggttactcaggctctttggactgggcctggcttcctgcctctcttctcctcccctcttgccccagtgtactcctcagcttggccaccctgagctgctttccacttttcacacaaaccaaatcatctttttcctctgcgctgactgctccttcttcctcggtgcctgactagccctcacgcatccttccgtgactcagcttacactcttttcttccagaaacctctgcctgggccccaaggttgggtgagataaggctcgatgccccttatcagtgctcctgtgacatatgctactttccccagcagcaggtgtttgctggcactgtcattgcccgttagctgcttctgctacagtgtgaactctgccaggatggaaatatggctgctgggttcacagctggatccccagcaccagactgtgccaagaatagaatatgtgcttaaaagatggtcagagaattcagcaacgttccctgagggtcatacagtctagaaatacacacaagacccagactagatgcacatatagatagccctggaaaaataagaaaggttaaaacggataataaggctatttggtggctacgacgagtttattagagagggcaaccggagcccccagccccactcacggacgttctctcaaatccacctctgaaggtgcatgagataaaggagagctatttactgcacctagacgccaggcaatgaaaacggggtgagggtcgagggcagggatctgaggagccacatcaggccagccttaccttcatgttgtcaggggggtctccttggcctgtagttgcacaaacaaatatcaccaggggctcgttaatcagattcacctcaacaaaaagacacacacacgtaagacctccggctgtaaggaccgggcgtccttcccaaaacttgacccccttctcctttactcaggctgacagggcagagggatttatgtgagcagccagaccgtcttctctttcaggttccaacctcctccaggtgctaaactgaactccccttcccaaacctccttccctaaatccgccataagggaaagacccgctctcagagaaaatgcgacgccttttgggtcgatgaccccgcgaggcgggcacgccaggcctgctcgtcccccacgccgaggccctagcgagccctcaccaccgggtaggagtccagggcctgcacccggcagccaagccgccggcgccgggcctcgcgacccagtctctccgacacatcctgagccgtgcctgtctggctgccgaagagcaccagaagctgcgggctcggcatcggggtgcgcccggtggtctggtctgagactaaaaactagaaggcagttcccgccggccgggttgcagggaccgctccgccttccgccttccgccgtcgaccggaagtgacgcactagggacgcgccctgtgggggcatggcgtccgatcgaggcgggcgttcacgggcggccagggttgagtcccgggtcggggccgggggattgccggcgcatcagggccgagggctggggctggcgg

SEQ ID NO: 4 - DNA, 225 bp, Homo sapiens
ggcgtccgatcgaggcgggcgttcacgggcggccagggttgagtcccgggtcggggccgggggattgccggcgcatcagggccgagggctggggctggcggggccgctcgctgcctctcgctcgcagcagcggcggcaggcgcgggcgagggccacggggagaggagacgcagccccgcgggtggcacgctcggccgggccccggcccgcgctcaacgggcgcga

SEQ ID NO: 5 - DNA, 941 bp, Homo sapiens
cctcaccgaggtgccggagagggagcgctggacaactagaatgttgacctcgagccgaggccctacttgcagcgcaccggaggagaggctctctagtctgaaggcaccgccggcttgcgccgagctgagtgccgggtttccctattccaatcctgtttgaaatggtttcttcagcagggcttaaaagagcagccttcatcctgaaaatgtatttccttttgtttaatgctttgagtagataatcctgaattgaggtcatgaggaggccccccaggccagacagtcctgaacccctctgacacttggaaactgaatataagtaaaatgtccaggtggactctgagtatttcctgtggatcctgggaaagtactgttgcacaaaggctgcaaagctggactcaggaatgtcctccaaccagcagcgctgacctaagagctccctgtgccgtctatccagaccagacttcggtagatgcctttgttagatctatcacatgtaaacgagcttgtatctccttccctgtgccacgagagagattggctttttattccagtctaggcagagacagaagaatgttgaataagagcacgattagagtcctgtctggttatctgttgcccaagaaaagaactctgctgtccaggcactgcttggcttactatcccagcaaagactgcagttttgtggacttttgaccaccttgggctggcactcttagcacacctgagacagatttaagcctccctaagagactgaagagaggaacaggtgtcagatactcataggcactgagatctacaaatgggaagcttgtgagtggcccatctttgttggcctacgaactttggtttgatgccagtcaggtgccacatgagaacctttgctgagatgcaaataaagtaagagaatgttttcctgaaaaaaaaaaaaaaaaaaaaaa

SEQ ID NO: 6 - protein, 12 aa, Homo sapiens
MLFSLRELVQWL

SEQ ID NO: 7 - protein, 14 aa, Homo sapiens
RVDGLVPGLSWWNV

SEQ ID NO: 8 - protein, 6 aa, Homo sapiens
QDGEKR

SEQ ID NO: 9 - protein, 9 aa, Homo sapiens
CQKLAEQTR

SEQ ID NO: 10 - protein, 6 aa, Homo sapiens
RACRVN

SEQ ID NO: 11 - DNA, 840 bp, Mus musculus
gatagcatctggacaccacgggcttctgctagcctccggttccgggtccggggttggggtcctcagggtcgcagagccccagggccggcgtccaggccaggttgggctgcttgctgcctcccgtgtgcagcggcggcagcaggcggtcgccaggtctgcggtcggagacgtcacagcccggtgcggggcaccggtggccggtgttaagcaggcgcgatgttattctcgctgcgggagctggtgcagtggctgggcttcgccacctttgagatattcgtgcacctgctggccctgttggtgttctccgtactgttggcactgcgagtggatggcttgactccgggcctctcctggtggaacgtctttgtgccctttttcgccgccgacgggctcagtacctacttcaccaccatcgtttccgttcgactcttccaagatggggagaagcgactggctgtgctgcgcctcttctgggttctcaccgtccttagcctcaagtttgtctttgagatgttgctgtgccagaagctagtggagcagagctcgagagctctggttcggcctgatcacgtctccggtcttcattctcctgcagctgctcatgatccgggcttgtcgcgtcaactagcctcttgcagtggctggaaatggagcactgcgcagctggagttctggacctcccggtcctgacccaacttggtgccacactggtggagctttggtcagaaatcagtgtttgcttgtgccccagtttactgtccagttttctttatatataagatcgtttcctcgagcaaagcttaaaagtgaagtcttgtttattaaaacttatttccagtt

SEQ ID NO: 12 - DNA, 797 bp, Mus musculus
acgggcttctgctagcctccggttccgggtccggggttggggtcctcagggtcgcagagccccagggccggcgtccaggccaggttgggctgcttgctgcctcccgtgtgcagcggcggcagcaggcggtcgccaggtctgcggtcggagacgtcacagcccggtgcggggcaccggtggccggtgttaagcaggcgcgatgttattctcgctgcgggagctggtgcagtggctgggcttcgccacctttgagatattcgtgcacctgctggccctgttggtgttctccgtactgttggcactgcgagtggatggcttgactccgggcctctcctggtggaacgtctttgtgccctttttcgccgccgacgggctcagtacctacttcaccaccatcgtttccgttcgactcttccaagatggggagaagcgactggctgtgctgcgcctcttctgggttctcaccgtccttagcctcaagtttgtctttgagatgttgctgtgccagaagctagtggagcagactcgagagctctggttcggcctgatcacgtctccggtcttcattctcctgcagctgctcatgatccgggcttgtcgcgtcaactagcctcttgcagtggctggaaatggagcactgcgcagctggagttctggacctcccggtcctgacccaacttggtgccacactggtggagctttggtcagaaatcagtgtttgcttgtgcccagtttactgtccagttttctttatatataagatcgtttcctcgagcaaaaaaaaaaaaaaaaaaaaa

SEQ ID NO: 13 - protein, 157 aa, Mus musculus
MLFSLRELVQWLGFATFEIFVHLLALLVFSVLLALRVDGLTPGLSWWNVFVPFFAADGLSTYFTTIVSVRLFQDGEKRLAVLRLFWVLTVLSLKFVFEMLLCQKLVEQSSRALVRPDHVSGLHSPAAAHDPGLSRQLASCSGWKWSTAQLEFWTSRS

SEQ ID NO: 14 - protein, 136 aa, Mus musculus
MLFSLRELVQWLGFATFEIFVHLLALLVFSVLLALRVDGLTPGLSWWNVFVPFFAADGLSTYFTTIVSVRLFQDGEKRLAVLRLFWVLTVLSLKFVFEMLLCQKLVEQTRELWFGLITSPVFILLQLLMIRACRVN

SEQ ID NO: 15 - protein, 136 aa, Rattus norvegicus
MLFSLRELVQWLGFATFEIFVHLLALLVFSVLLALRVDGLAPGLSWWNVFVPFFAADGLSTYFTTIVSVRLFQDGEKRLAVLRLFWVLTVLSLKFVFEMLLCQKLVEQTRELWFGLITSPVFILLQLLMIRACRVN

SEQ ID NO: 16 - protein, 136 aa, Canis familiaris; identical to SEQ ID NO: 15

SEQ ID NO: 17 - protein, 136 aa, Pan troglodytes; identical to SEQ ID NO: 2

SEQ ID NO: 18 - protein, 136 aa, Xenopus tropicalis
MLFSLLELVQWLGFAQLEIFLHIWALLVFTVLLALKADGFAPDMSWWNIFIPFFTADGLSTYFTTIVTVRLFQDGEKRQAVLRLFWILTILSLKFVFEMLLCQKLVEQSRELWFGLIMSPVFILLQLLMIRACRVN

SEQ ID NO: 19 - protein, 140 aa, Danio rerio
MLFSLRELVQWLGFATFELFLHLGALLVFSVLVALHMDVDKQTLEMSWWLVFSPLFTADGLSTYFTAIVSIRLYQEGEKRLAVLRLLWVLTVLSLKLVCEVLLCQKLAEQERAQDLWFGLIVSPLFILLQLLMIRACRVN

SEQ ID NO: 20 - protein, 140 aa, Gallus gallus
MLFSLRELVQWLGFATFEIFLHGLALLVFSVLLVLKVDSEASPLSWWVVFVPFFVADGLSTYFTAIVSVRLFQDGEKRLAVLRLFWILTILSLKFVFEMLLCQKLVERTRAVVRTHHVARLHPSAAPHDQGLPRELSLRG

SEQ ID NO: 21 - protein, 135 aa, Branchiostoma floridae
MLFSLKEIIQWIGLSAFELWLHVVSLLVFSILLALKLEGVLSTTWWTVFIPLFASDGLHAYFSSIVFLRLNLEGDLRTAGIRTAWSAVVLVLLFAFKMLLCQKLEDQNNLTFAMIMSPVFILLQVLMIRACQVGN

SEQ ID NO: 22 - DNA, 21 bp, Artificial
  siRNA guide sequence (5′ -> 3′)
uaguaagccaagcagugcctg

SEQ ID NO: 23 - DNA, 21 bp, Artificial
  siRNA guide sequence (5′ -> 3′)
uugugcaacaguacuuuccca

SEQ ID NO: 24 - DNA, 21 bp, Artificial
  siRNA guide sequence (5′ -> 3′)
uuacuuauauucaguuuccaa

SEQ ID NO: 25 - DNA, 21 bp, Artificial
  siRNA guide sequence (5′ -> 3′)
uaugaguaucugacaccugtt

SEQ ID NO: 26 - DNA, 21 bp, Artificial
  siRNA guide sequence (5′ -> 3′)
uaagagugccagcccaaggtg

SEQ ID NO: 27 - DNA, 21 bp, Artificial
  siRNA guide sequence (5′ -> 3′)
uaguugacccgacaggcgcgg

SEQ ID NO: 28 - DNA, 21 bp, Artificial
  siRNA guide sequence (5′ -> 3′)
ucguaggccaacaaagauggg

SEQ ID NO: 29 - DNA, 21 bp, Artificial
  siRNA guide sequence (5′ -> 3′)
ucuugggcaacagauaaccag

SEQ ID NO: 30 - DNA, 21 bp, Artificial
  siRNA guide sequence (5′ -> 3′)
ugauagaucuaacaaaggcat

SEQ ID NO: 31 - DNA, 21 bp, Artificial
  siRNA guide sequence (5′ -> 3′)
ucuuagggaggcuuaaauctg

SEQ ID NO: 32 - RNA, 21 nt, Artificial
  siRNA guide sequence (5′ -> 3′)
uuggaauagggaaacccggca

SEQ ID NO: 33 - DNA, 21 bp, Artificial
  siRNA guide sequence (5′ -> 3′)
uaguuguccagcgcucccuct

SEQ ID NO: 34 - DNA, 21 bp, Artificial
  siRNA guide sequence (5′ -> 3′)
uucucauguggcaccugactg

SEQ ID NO: 35 - RNA, 21 nt, Artificial
  siRNA guide sequence (5′ -> 3′)
ugguuggaggacauuccugag

SEQ ID NO: 36 - RNA, 21 nt, Artificial
  siRNA guide sequence (5′ -> 3′)
uuaucuacucaaagcauuaaa

SEQ ID NO: 37 - RNA, 21 nt, Artificial
  siRNA guide sequence (5′ -> 3′)
uucuggcacaacagcaucucg

SEQ ID NO: 38 - RNA, 21 nt, Artificial
  siRNA guide sequence (5′ -> 3′)
uuccaccaggagaggcccggg

SEQ ID NO: 39 - RNA, 21 nt, Artificial
  siRNA guide sequence (5′ -> 3′)
ucuaaucgugcucuuauucaa

SEQ ID NO: 40 - RNA, 21 nt, Artificial
  siRNA guide sequence (5′ -> 3′)
uuacuuuauuugcaucucagc

SEQ ID NO: 41 - RNA, 21 nt, Artificial
  siRNA guide sequence (5′ -> 3′)
ugaacgcccgccucgaucgga

SEQ ID NO: 42 - RNA, 21 nt, Artificial
  siRNA guide sequence (5′ -> 3′)
uacuuucccaggauccacagg

SEQ ID NO: 43 - DNA, 21 bp, Artificial
  siRNA guide sequence (5′ -> 3′)
auacaagcucguuuacaugtg

SEQ ID NO: 44 - RNA, 21 nt, Artificial
  siRNA guide sequence (5′ -> 3′)
uacaagcucguuuacauguga

SEQ ID NO: 45 - RNA, 21 nt, Artificial
  siRNA guide sequence (5′ -> 3′)
uacaugugauagaucuaacaa

SEQ ID NO: 46 - RNA, 21 nt, Artificial
  siRNA guide sequence (5′ -> 3′)
ugugcaacaguacuuucccag

SEQ ID NO: 47 - DNA, 21 bp, Artificial
  siRNA guide sequence (5′ -> 3′)
ucgaggucaacauucuagutg

SEQ ID NO: 48 - DNA, 21 bp, Artificial
  siRNA guide sequence (5′ -> 3′)
ucuaguuguccagcgcuccct

SEQ ID NO: 49 - DNA, 21 bp, Artificial
  siRNA guide sequence (5′ -> 3′)
auuuguagaucucagugccta

SEQ ID NO: 50 - RNA, 21 nt, Artificial
  siRNA guide sequence (5′ -> 3′)
uuugcagccuuugugcaacag

SEQ ID NO: 51 - RNA, 21 nt, Artificial
  siRNA guide sequence (5′ -> 3′)
ucaaacaggauuggaauaggg

SEQ ID NO: 52 - RNA, 21 nt, Artificial
  siRNA guide sequence (5′ -> 3′)
ugcaacaguacuuucccagga

SEQ ID NO: 53 - DNA, 21 bp, Artificial
  siRNA guide sequence (5′ -> 3′)
cuuauauucaguuuccaagtg

SEQ ID NO: 54 - RNA, 21 nt, Artificial
  siRNA guide sequence (5′ -> 3′)
aaaggcacgaacacguuccac

SEQ ID NO: 55 - DNA, 21 bp, Artificial
  siRNA guide sequence (5′ -> 3′)
uuuguagaucucagugccuat

SEQ ID NO: 56 - DNA, 21 bp, Artificial
  siRNA guide sequence (5′ -> 3′)
aaaggcaucuaccgaaguctg

SEQ ID NO: 57 - RNA, 21 nt, Artificial
  siRNA guide sequence (5′ -> 3′)
uucuugggcaacagauaacca

SEQ ID NO: 58 - DNA, 21 bp, Artificial
  siRNA guide sequence (5′ -> 3′)
ugcugguuggaggacauucct

SEQ ID NO: 59 - RNA, 21 nt, Artificial
  siRNA guide sequence (5′ -> 3′)
uuucaaacaggauuggaauag

SEQ ID NO: 60 - DNA, 21 bp, Artificial
  siRNA guide sequence (5′ -> 3′)
uugcaucucagcaaagguuct

SEQ ID NO: 61 - DNA, 21 bp, Artificial
  siRNA guide sequence (5′ -> 3′)
aaucgugcucuuauucaacat

SEQ ID NO: 62 - DNA, 21 bp, Artificial
  siRNA complement  (5′ -> 3′)
ggcacugcuuggcuuacuatt

SEQ ID NO: 63 - DNA, 21 bp, Artificial
  siRNA complement  (5′ -> 3′)
ggaaaguacuguugcacaatt

SEQ ID NO: 64 - DNA, 21 bp, Artificial
  siRNA complement  (5′ -> 3′)
ggaaacugaauauaaguaatt

SEQ ID NO: 65 - DNA, 21 bp, Artificial
  siRNA complement  (5′ -> 3′)
caggugucagauacucauatt

SEQ ID NO: 66 - DNA, 21 bp, Artificial
  siRNA complement  (5′ -> 3′)
ccuugggcuggcacucuuatt

SEQ ID NO: 67 - DNA, 21 bp, Artificial
  siRNA complement  (5′ -> 3′)
gcgccugucgggucaacuatt

SEQ ID NO: 68 - DNA, 21 bp, Artificial
  siRNA complement  (5′ -> 3′)
caucuuuguuggccuacgatt

SEQ ID NO: 69 - DNA, 21 bp, Artificial
  siRNA complement  (5′ -> 3′)
gguuaucuguugcccaagatt

SEQ ID NO: 70 - DNA, 21 bp, Artificial
  siRNA complement  (5′ -> 3′)
gccuuuguuagaucuaucatt

SEQ ID NO: 71 - DNA, 21 bp, Artificial
  siRNA complement  (5′ -> 3′)
gauuuaagccucccuaagatt

SEQ ID NO: 72 - DNA, 21 bp, Artificial
  siRNA complement  (5′ -> 3′)
ccggguuucccuauuccaatt

SEQ ID NO: 73 - DNA, 21 bp, Artificial
  siRNA complement  (5′ -> 3′)
agggagcgcuggacaacuatt

SEQ ID NO: 74 - DNA, 21 bp, Artificial
  siRNA complement  (5′ -> 3′)
gucaggugccacaugagaatt

SEQ ID NO: 75 - DNA, 21 bp, Artificial
  siRNA complement  (5′ -> 3′)
caggaauguccuccaaccatt

SEQ ID NO: 76 - DNA, 21 bp, Artificial
  siRNA complement  (5′ -> 3′)
uaaugcuuugaguagauaatt

SEQ ID NO: 77 - DNA, 21 bp, Artificial
  siRNA complement  (5′ -> 3′)
agaugcuguugugccagaatt

SEQ ID NO: 78 - DNA, 21 bp, Artificial
  siRNA complement  (5′ -> 3′)
cgggccucuccugguggaatt

SEQ ID NO: 79 - DNA, 21 bp, Artificial
  siRNA complement  (5′ -> 3′)
gaauaagagcacgauuagatt

SEQ ID NO: 80 - DNA, 21 bp, Artificial
  siRNA complement  (5′ -> 3′)
ugagaugcaaauaaaguaatt

SEQ ID NO: 81 - DNA, 21 bp, Artificial
  siRNA complement  (5′ -> 3′)
cgaucgaggcgggcguucatt

SEQ ID NO: 82 - DNA, 21 bp, Artificial
  siRNA complement  (5′ -> 3′)
uguggauccugggaaaguatt

SEQ ID NO: 83 - DNA, 21 bp, Artificial
  siRNA complement  (5′ -> 3′)
cauguaaacgagcuuguautt

SEQ ID NO: 84 - DNA, 21 bp, Artificial
  siRNA complement  (5′ -> 3′)
acauguaaacgagcuuguatt

SEQ ID NO: 85 - DNA, 21 bp, Artificial
  siRNA complement  (5′ -> 3′)
guuagaucuaucacauguatt

SEQ ID NO: 86 - DNA, 21 bp, Artificial
  siRNA complement  (5′ -> 3′)
gggaaaguacuguugcacatt

SEQ ID NO: 87 - DNA, 21 bp, Artificial
  siRNA complement  (5′ -> 3′)
acuagaauguugaccucgatt

SEQ ID NO: 88 - DNA, 21 bp, Artificial
  siRNA complement  (5′ -> 3′)
ggagcgcuggacaacuagatt

SEQ ID NO: 89 - DNA, 21 bp, Artificial
  siRNA complement  (5′ -> 3′)
ggcacugagaucuacaaautt

SEQ ID NO: 90 - DNA, 21 bp, Artificial
  siRNA complement  (5′ -> 3′)
guugcacaaaggcugcaaatt

SEQ ID NO: 91 - DNA, 21 bp, Artificial
  siRNA complement  (5′ -> 3′)
cuauuccaauccuguuugatt

SEQ ID NO: 92 - DNA, 21 bp, Artificial
  siRNA complement  (5′ -> 3′)
cugggaaaguacuguugcatt

SEQ ID NO: 93 - DNA, 21 bp, Artificial
  siRNA complement  (5′ -> 3′)
cuuggaaacugaauauaagtt

SEQ ID NO: 94 - DNA, 21 bp, Artificial
  siRNA complement  (5′ -> 3′)
ggaacguguucgugccuuutt

SEQ ID NO: 95 - DNA, 21 bp, Artificial
  siRNA complement  (5′ -> 3′)
aggcacugagaucuacaaatt

SEQ ID NO: 96 - DNA, 21 bp, Artificial
  siRNA complement  (5′ -> 3′)
gacuucgguagaugccuuutt

SEQ ID NO: 97 - DNA, 21 bp, Artificial
  siRNA complement  (5′ -> 3′)
guuaucuguugcccaagaatt

SEQ ID NO: 98 - DNA, 21 bp, Artificial
  siRNA complement  (5′ -> 3′)
gaauguccuccaaccagcatt

SEQ ID NO: 99 - DNA, 21 bp, Artificial
  siRNA complement  (5′ -> 3′)
auuccaauccuguuugaaatt

SEQ ID NO: 100 - DNA, 21 bp, Artificial
  siRNA complement  (5′ -> 3′)
aaccuuugcugagaugcaatt

SEQ ID NO: 101 - DNA, 21 bp, Artificial
  siRNA complement  (5′ -> 3′)
guugaauaagagcacgauutt

SEQ ID NO: 102 - DNA, 19 bp, Artificial
  shDNA target sequence
gcgcctgtcgggtcaacta

SEQ ID NO: 103 - DNA, 56 bp, Artificial
  Sense DNA oligos
ccgggcgcctgtcgggtcaactattcaagagatagttgacccgacaggcgcttttt

SEQ ID NO: 104 - DNA, 56 bp, Artificial
  Antisense DNA oligos
aattaaaaagcgcctgtcgggtcaactatctcttgaatagttgacccgacaggcgc

SEQ ID NO: 105 - DNA, 19 bp, Artificial
  shDNA target sequence
gcctttgttagatctatca

SEQ ID NO: 106 - DNA, 56 bp, Artificial
  Sense DNA oligo
ccgggcctttgttagatctatcattcaagagatgatagatctaacaaaggcttttt

SEQ ID NO: 107 - DNA, 56 bp, Artificial
  Antisense DNA oligo
aattaaaaagcctttgttagatctatcatctcttgaatgatagatctaacaaaggc

SEQ ID NO: 108 - DNA, 19 bp, Artificial
  shDNA target sequence
catctttgttggcctacga

SEQ ID NO: 109 - DNA, 56 bp, Artificial
  Sense DNA oligo
ccggcatctttgttggcctacgattcaagagatcgtaggccaacaaagatgttttt

SEQ ID NO: 110 - DNA, 56 bp, Artificial
  Antisense DNA oligo
aattaaaaacatctttgttggcctacgatctcttgaatcgtaggccaacaaagatg

SEQ ID NO: 111 - DNA, 19 bp, Artificial
  shDNA target sequence
ggttatctgttgcccaaga

SEQ ID NO: 112 - DNA, 56 bp, Artificial
  Sense DNA oligo
ccggggttatctgttgcccaagattcaagagatcttgggcaacagataaccttttt

SEQ ID NO: 113 - DNA, 56 bp, Artificial
  Antisense DNA oligo
aattaaaaaggttatctgttgcccaagatctcttgaatcttgggcaacagataacc

SEQ ID NO: 114 - DNA, 19 bp, Artificial
  shDNA target sequence
gatttaagcctccctaaga

SEQ ID NO: 115 - DNA, 56 bp, Artificial
  Sense DNA oligo
ccgggatttaagcctccctaagattcaagagatcttagggaggcttaaatcttttt

SEQ ID NO: 116 - DNA, 56 bp, Artificial
  Antisense DNA oligo
aattaaaaagatttaagcctccctaagatctcttgaatcttagggaggcttaaatc

SEQ ID NO: 117 - DNA, 19 bp, Artificial
  shDNA target sequence
actagaatgttgacctcga

SEQ ID NO: 118 - DNA, 56 bp, Artificial
  Sense DNA oligo
ccggactagaatgttgacctcgattcaagagatcgaggtcaacattctagtttttt

SEQ ID NO: 119 - DNA, 56 bp, Artificial
  Antisense DNA oligo
aattaaaaaactagaatgttgacctcgatctcttgaatcgaggtcaacattctagt

SEQ ID NO: 120 - DNA, 19 bp, Artificial
  shDNA target sequence
ccgggtttccctattccaa

SEQ ID NO: 121 - DNA, 56 bp, Artificial
  Sense DNA oligo
ccggccgggtttccctattccaattcaagagattggaatagggaaacccggttttt

SEQ ID NO: 122 - DNA, 56 bp, Artificial
  Antisense DNA oligo
aattaaaaaccgggtttccctattccaatctcttgaattggaatagggaaacccgg

SEQ ID NO: 123 - DNA, 19 bp, Artificial
  shDNA target sequence
gtcaggtgccacatgagaa

SEQ ID NO: 124 - DNA, 56 bp, Artificial
  Sense DNA oligo
ccgggtcaggtgccacatgagaattcaagagattctcatgtggcacctgacttttt

SEQ ID NO: 125 - DNA, 56 bp, Artificial
  Antisense DNA oligo
aattaaaaagtcaggtgccacatgagaatctcttgaattctcatgtggcacctgac

SEQ ID NO: 126 - DNA, 19 bp, Artificial
  shDNA target sequence
caggaatgtcctccaacca

SEQ ID NO: 127 - DNA, 56 bp, Artificial
  Sense DNA oligo
ccggcaggaatgtcctccaaccattcaagagatggttggaggacattcctgttttt

SEQ ID NO: 128 - DNA, 56 bp, Artificial
  Antisense DNA oligo
aattaaaaacaggaatgtcctccaaccatctcttgaatggttggaggacattcctg

SEQ ID NO: 129 - DNA, 21 bp, Artificial
  shDNA target sequence
ccttgggcuggcactcttatt

SEQ ID NO: 130 - DNA, 56 bp, Artificial
  Sense DNA oligo
ccggccttgggcuggcactcttattcaagagataagagtgccagcccaaggttttt

SEQ ID NO: 131 - DNA, 56 bp, Artificial
  Antisense DNA oligo
aattaaaaaccttgggctggcactcttatctcttgaataagagtgccagcccaagg

SEQ ID NO: 132 - DNA, 19 bp, Artificial
  shDNA target sequence
ggtagaaagtcgggacaaa

SEQ ID NO: 133 - DNA, 56 bp, Artificial
  Sense DNA oligo
ccggggtagaaagtcgggacaaattcaagagatttgtcccgactttctaccttttt

SEQ ID NO: 134 - DNA, 56 bp, Artificial
  Antisense DNA oligo
aattaaaaaggtagaaagtcgggacaaatctcttgaatttgtcccgactttctacc

SEQ ID NO: 135 - DNA, 19 bp, Artificial
  shDNA target sequence
cttacgctgagtacttcga

SEQ ID NO: 136 - DNA, 56 bp, Artificial
  Sense DNA oligo
ccggcttacgctgagtacttcgattcaagagatcgaagtactcagcgtaagttttt

SEQ ID NO: 137 - DNA, 56 bp, Artificial
  Antisense DNA oligo
aattaaaaacttacgctgagtacttcgatctcttgaatcgaagtactcagcgtaag